SEQUENCE LISTING (81 sequences)

SEQ ID NO: 1 - DNA, 21 bp, Artificial Sequence
  Probe of katG: probe
ctcgatgccgctggtgatcgc

SEQ ID NO: 2 - DNA, 21 bp, Artificial Sequence
  Probe of katG: probe
gcgatcaccaccggcatcgag

SEQ ID NO: 3 - DNA, 21 bp, Artificial Sequence
  Probe of inhA: probe
ggcgagacgataggttgtcgg

SEQ ID NO: 4 - DNA, 21 bp, Artificial Sequence
  Probe of inhA: probe
ggcgagatgataggttgtcgg

SEQ ID NO: 5 - DNA, 23 bp, Artificial Sequence
  Probe of rpoB: probe
gccagctgagccaattcatggac

SEQ ID NO: 6 - DNA, 24 bp, Artificial Sequence
  Probe of rpoB: probe
gccaattcatggaccagaacaacc

SEQ ID NO: 7 - DNA, 22 bp, Artificial Sequence
  Probe of rpoB: probe
tggaccagaacaacccgctgtc

SEQ ID NO: 8 - DNA, 22 bp, Artificial Sequence
  Probe of rpoB: probe
acaacccgctgtcggggttgac

SEQ ID NO: 9 - DNA, 21 bp, Artificial Sequence
  Probe of rpoB: probe
ggttgacccacaagcgccgac

SEQ ID NO: 10 - DNA, 23 bp, Artificial Sequence
  Probe of rpoB: probe
cgactgtcggcactggggcccgg

SEQ ID NO: 11 - DNA, 23 bp, Artificial Sequence
  Probe of rpoB: probe
cagccagccagctgagccaattc

SEQ ID NO: 12 - DNA, 23 bp, Artificial Sequence
  Probe of rpoB: probe
ccagccagctgagccaattcatg

SEQ ID NO: 13 - DNA, 23 bp, Artificial Sequence
  probe
agctgagccaattcatggaccag

SEQ ID NO: 14 - DNA, 24 bp, Artificial Sequence
  Probe of rpoB: probe
tgagccaattcatggaccagaaca

SEQ ID NO: 15 - DNA, 24 bp, Artificial Sequence
  Probe of rpoB: probe
aattcatggaccagaacaacccgc

SEQ ID NO: 16 - DNA, 23 bp, Artificial Sequence
  Probe of rpoB: probe
tcatggaccagaacaacccgctg

SEQ ID NO: 17 - DNA, 22 bp, Artificial Sequence
  Probe of rpoB: probe
accagaacaacccgctgtcggg

SEQ ID NO: 18 - DNA, 22 bp, Artificial Sequence
  Probe of rpoB: probe
agaacaacccgctgtcggggtt

SEQ ID NO: 19 - DNA, 21 bp, Artificial Sequence
  Probe of rpoB: probe
acccgctgtcggggttgaccc

SEQ ID NO: 20 - DNA, 22 bp, Artificial Sequence
  Probe of rpoB: probe
cgctgtcggggttgacccacaa

SEQ ID NO: 21 - DNA, 22 bp, Artificial Sequence
  Probe of rpoB: probe
tgtcggggttgacccacaagcg

SEQ ID NO: 22 - DNA, 21 bp, Artificial Sequence
  Probe of rpoB: probe
cggggttgacccacaagcgcc

SEQ ID NO: 23 - DNA, 22 bp, Artificial Sequence
  Probe of rpoB: probe
tgacccacaagcgccgactgtc

SEQ ID NO: 24 - DNA, 21 bp, Artificial Sequence
  Probe of rpoB: probe
cccacaagcgccgactgtcgg

SEQ ID NO: 25 - DNA, 21 bp, Artificial Sequence
  Probe of rpoB: probe
acaagcgccgactgtcggcgc

SEQ ID NO: 26 - DNA, 21 bp, Artificial Sequence
  Probe of rpoB: probe
agcgccgactgtcggcgctgg

SEQ ID NO: 27 - DNA, 21 bp, Artificial Sequence
  Probe of rpoB: probe
gccgactgtcggcgctggggc

SEQ ID NO: 28 - DNA, 21 bp, Artificial Sequence
  Probe of rpoB: probe
gactgtcggcgctggggcccg

SEQ ID NO: 29 - DNA, 20 bp, Artificial Sequence
  Probe of rpoB: probe
tgtcggcgctggggcccggc

SEQ ID NO: 30 - DNA, 20 bp, Artificial Sequence
  Probe of rpoB: probe
cggcgctggggcccggcggt

SEQ ID NO: 31 - DNA, 20 bp, Artificial Sequence
  Probe of rpoB: probe
cgctggggcccggcggtctg

SEQ ID NO: 32 - DNA, 21 bp, Artificial Sequence
  Probe of rpoB: probe
tggggcccggcggtctgtcac

SEQ ID NO: 33 - DNA, 21 bp, Artificial Sequence
  Forward primer for katG: primer
cgctggagcagatgggcttgg

SEQ ID NO: 34 - DNA, 21 bp, Artificial Sequence
  Reverse primer for katG: primer
gtcagctcccactcgtagccg

SEQ ID NO: 35 - DNA, 21 bp, Artificial Sequence
  Forward primer for inhA: primer
gcagccacgttacgctcgtgg

SEQ ID NO: 36 - DNA, 21 bp, Artificial Sequence
  Reverse primer for inhA: primer
cgatcccccggtttcctccgg

SEQ ID NO: 37 - DNA, 20 bp, Artificial Sequence
  Forward primer for rpoB: primer
ggtcggcatgtcgcggatgg

SEQ ID NO: 38 - DNA, 21 bp, Artificial Sequence
  Reverse primer for rpoB: primer
gtagtgcgacgggtgcacgtc

SEQ ID NO: 39 - DNA, 10 bp, Mycobacterium tuberculosis
  target
accagcggca

SEQ ID NO: 40 - DNA, 10 bp, Mycobacterium tuberculosis
  target
gccggtggtg

SEQ ID NO: 41 - DNA, 10 bp, Mycobacterium tuberculosis
  target
tatcgtctcg

SEQ ID NO: 42 - DNA, 10 bp, Mycobacterium tuberculosis
  target
tatcatctcg

SEQ ID NO: 43 - DNA, 10 bp, Mycobacterium tuberculosis
  target
gaattggctc

SEQ ID NO: 44 - DNA, 10 bp, Mycobacterium tuberculosis
  target
ctggtccatg

SEQ ID NO: 45 - DNA, 10 bp, Mycobacterium tuberculosis
  target
ggttgttctg

SEQ ID NO: 46 - DNA, 10 bp, Mycobacterium tuberculosis
  target
cccgacagcg

SEQ ID NO: 47 - DNA, 10 bp, Mycobacterium tuberculosis
  target
gcttgtgggt

SEQ ID NO: 48 - DNA, 10 bp, Mycobacterium tuberculosis
  target
ccagtgccga

SEQ ID NO: 49 - DNA, 15 bp, Mycobacterium tuberculosis
  target
atcaccagcggcatc

SEQ ID NO: 50 - DNA, 15 bp, Mycobacterium tuberculosis
  target
gatgccggtggtgta

SEQ ID NO: 51 - DNA, 15 bp, Mycobacterium tuberculosis
  target
acctatcgtctcgcc

SEQ ID NO: 52 - DNA, 15 bp, Mycobacterium tuberculosis
  target
acctatcatctcgcc

SEQ ID NO: 53 - DNA, 15 bp, Mycobacterium tuberculosis
  target
atgaattggctcagc

SEQ ID NO: 54 - DNA, 15 bp, Mycobacterium tuberculosis
  target
gttctggtccatgaa

SEQ ID NO: 55 - DNA, 15 bp, Mycobacterium tuberculosis
  target
gcgggttgttctggt

SEQ ID NO: 56 - DNA, 15 bp, Mycobacterium tuberculosis
  target
aaccccgacagcggg

SEQ ID NO: 57 - DNA, 15 bp, Mycobacterium tuberculosis
  target
ggcgcttgtgggtca

SEQ ID NO: 58 - DNA, 15 bp, Mycobacterium tuberculosis
  target
gccccagtgccgaca

SEQ ID NO: 59 - DNA, 10 bp, Artificial Sequence
  Probe of katG: probe
tgccgctggt

SEQ ID NO: 60 - DNA, 10 bp, Artificial Sequence
  Probe of katG: probe
caccaccggc

SEQ ID NO: 61 - DNA, 10 bp, Artificial Sequence
  Probe of inhA: probe
cgagacgata

SEQ ID NO: 62 - DNA, 10 bp, Artificial Sequence
  Probe of inhA: probe
cgagatgata

SEQ ID NO: 63 - DNA, 10 bp, Artificial Sequence
  Probe of rpoB: probe
gagccaattc

SEQ ID NO: 64 - DNA, 10 bp, Artificial Sequence
  Probe of rpoB: probe
catggaccag

SEQ ID NO: 65 - DNA, 10 bp, Artificial Sequence
  Probe of rpoB: probe
cagaacaacc

SEQ ID NO: 66 - DNA, 10 bp, Artificial Sequence
  Probe of rpoB: probe
cgctgtcggg

SEQ ID NO: 67 - DNA, 10 bp, Artificial Sequence
  Probe of rpoB: probe
acccacaagc

SEQ ID NO: 68 - DNA, 10 bp, Artificial Sequence
  Probe of rpoB: probe
tcggcactgg

SEQ ID NO: 69 - DNA, 15 bp, Artificial Sequence
  Probe of katG: probe
gatgccgctggtgat

SEQ ID NO: 70 - DNA, 15 bp, Artificial Sequence
  Probe of katG: probe
atcaccaccggcatc

SEQ ID NO: 71 - DNA, 15 bp, Artificial Sequence
  Probe of inhA: probe
ggcgagacgataggt

SEQ ID NO: 72 - DNA, 15 bp, Artificial Sequence
  Probe of inhA: probe
ggcgagatgataggt

SEQ ID NO: 73 - DNA, 17 bp, Artificial Sequence
  Probe of rpoB: probe
agctgagccaattcatg

SEQ ID NO: 74 - DNA, 18 bp, Artificial Sequence
  Probe of rpoB: probe
aattcatggaccagaaca

SEQ ID NO: 75 - DNA, 15 bp, Artificial Sequence
  Probe of rpoB: probe
accagaacaacccgc

SEQ ID NO: 76 - DNA, 16 bp, Artificial Sequence
  Probe of rpoB: probe
acccgctgtcggggtt

SEQ ID NO: 77 - DNA, 15 bp, Artificial Sequence
  Probe of rpoB: probe
tgacccacaagcgcc

SEQ ID NO: 78 - DNA, 17 bp, Artificial Sequence
  Probe of rpoB: probe
ctgtcggcactggggcc

SEQ ID NO: 79 - DNA, 21 bp, Artificial Sequence
  Complement of Probe 1: probe
gagctacggcgaccactagcg

SEQ ID NO: 80 - DNA, 21 bp, Artificial Sequence
  Complement of Probe 2: probe
cgctagtggtggccgtagctc

SEQ ID NO: 81 - DNA, 21 bp, Mycobacterium tuberculosis
  target
ccgggttgacccacaagcgcc

(19) United States
(12) Patent Application Publication
(10) Pub. No.: US 2010/0273146 A1
(43) Pub. Date: Oct. 28, 2010

(54) TB resistance assay

(76) Inventor: Timothy Brown, London (GB)

Correspondence Address: MCCRACKEN & FRANK LLP, 311 S. WACKER DRIVE, SUITE 2500, CHICAGO, IL, 60606, US

(21) Appl. No.: 11/502,676

(22) Filed: Aug. 11, 2006

Related U.S. Application Data
(63) Continuation-in-part of application No. PCT/GB05/00499, filed on Feb. 11, 2005.

(30) Foreign Application Priority Data
Feb. 11, 2004 (GB) GB 0403039.1

(51) Int. Cl. C12Q 1/68; C07H 21/04

(52) U.S. Cl. 435/6; 536/24.32

(57) ABSTRACT

A diagnostic test for detecting multi-drug resistant Mycobacterium sp., in particular Mycobacterium tuberculosis, in a sample is presented, including corresponding reagents, uses thereof and kits therefor.

This application claims priority to PCT/GB2005/000499, filed Feb. 11, 2005, and to GB 0403039.1, filed Feb. 11, 2004, the disclosures of which are hereby incorporated herein by reference in their respective entireties.

The present invention relates to a diagnostic assay for multi-drug resistant Mycobacterium sp., in particular Mycobacterium tuberculosis, and to reagents and kits therefor.

Mycobacterium tuberculosis and closely related species make up a small group of mycobacteria known as the Mycobacterium tuberculosis complex (MTC). This group comprises five species—M. tuberculosis, M. microti, M. bovis, M. caneti, and M. africanum—which are the causative agent in the majority of cases of Mycobacterium tuberculosis infection (TB) throughout the world.

M. tuberculosis is responsible for more than three million deaths a year worldwide. The WHO estimates that up to a third of the world's population is infected with Mycobacterium tuberculosis and globally someone dies of TB every 15 seconds. Other mycobacteria are also pathogenic in man and animals, for example M. avium subsp. paratuberculosis which causes Johne's disease in ruminants, M. bovis which causes tuberculosis in cattle, M. avium and M. intracellulare which cause tuberculosis in immunocompromised patients (eg. AIDS patients, and bone marrow transplant patients), and M. leprae which causes leprosy in humans. Another important mycobacterial species is M. vaccae.

Epidemiological data shows high incidence rates of Mycobacterium sp. infection, including MTC infection such as Mycobacterium tuberculosis infection, across all former Soviet Union countries. In Russia, a TB incidence of 90.7/100,000 was recorded in 2000. Similarly high rates of drug resistance, particularly multiple drug resistance caused by multiple drug resistant Mycobacterium tuberculosis (MDRTB), have been reported, contributing to low cure rates and compromising the effectiveness of TB programmes. Rates of MDRTB as high as 14% of newly diagnosed cases have been reported in Estonia. In 2000, rates of primary and secondary resistance to at least one drug in North Russia were reported to be 33% and 85%, respectively, in the Archangelsk oblast. High drug resistance rates were reported in Samara and St Petersburg. Conversely, although large institutional outbreaks of drug resistant and MDRTB have been reported in the USA, the UK and other European countries the overall MDRTB incidence and prevalence are low.

A number of factors have contributed to the problem of microbial resistance. One is the accumulation of mutations over time and the subsequent horizontal and vertical transfer of the mutated genes to other organisms. Thus, for a given pathogen, entire classes of antibiotics have been rendered inactive. A further factor has been the absence of a new class of antibiotics in recent years. The emergence of multiple drug-resistant pathogenic bacteria such as Mycobacterium sp., in particular those species of the MTC, such as Mycobacterium tuberculosis, represents a serious threat to public health and new forms of therapy are urgently required.

Multi-drug resistance in Mycobacterium sp. such as Mycobacterium tuberculosis (M. tuberculosis) is assessed in relation to two drugs—rifampin and isoniazid. Multidrug-resistant (MDR) strains which are defined as being resistant to INH and RIF are emerging and their involvement in several outbreaks has been reported. Currently available methods for detection of drug resistance include sputum microscopy, and growth based methods that measure growth of bacteria on solid or liquid media (usually Lowenstein-Jensen media) in the presence of drugs—“Drug Susceptibility Testing” (DST). A disadvantage of growth-based techniques is that they are limited by the growth rate of Mycobacterium sp. such as M. tuberculosis, which has a doubling time of 16 hours (compare E. coli, which has a doubling time of 20 minutes). Hence, it typically takes at least 10-14 days (typically 21-30 days after the primary culture has been isolated) to identify drug resistant Mycobacterium sp. by this method. The use of non-standardised methods also compromises the accuracy of DST.

The viability of Mycobacterium sp. such as M. tuberculosis after exposure to drugs (and hence, drug resistance) can also be monitored by studying the ability of mycobacteriophage to successfully infect the organism. Two methods have been described. In one method, Mycobacterium sp. such as M. tuberculosis are treated with a drug, and then phage are used to infect the drug-treated M. tuberculosis. Extracellular phage are destroyed before the M. tuberculosis are lysed, and the phage are enumerated on a lawn of rapidly growing mycobacteria. The number of plaques is proportional to the number of viable M. tuberculosis. In the second method, a luminescent phage is used to infect drug-exposed Mycobacterium sp. such as M. tuberculosis, the luminescence being proportional to the number of viable M. tuberculosis. A disadvantage of each of these methods is that they both take approximately 2 days for identification of drug resistant Mycobacterium sp.

At least 11 genes have been reported to be involved in the development of resistance to the main anti-TB drugs. The detection of rifampin resistance (RIF resistance) or isoniazid resistance (INH resistance), is of importance clinically and for public health TB control.

Resistance to rifampin and isoniazid is conferred by mutations in three genes. RIF resistance is generally associated with single nucleotide substitutions. Mutations in the 81 bp core region of the rpoB gene (encoding the β-subunit of RNA polymerase) are known to be responsible for over 90% of RIF resistance—approximately 60-70% of mutations are found within two codons, 531 and 526.

Mutations in two different genes are known to be responsible for resistance to isoniazid. In more than 75% of cases, INH resistance occurs due to substitutions in the katG gene (encoding catalase-peroxidase), particularly at codon 315 (AGC-ACC). More rarely, INH resistance is due to mutations in the inhA and ahpC genes.

The prevalence of mutations at codon 315 of the katG gene varies depending on the geographical region studied with percentages from 35% in Beirut to over 90% in Latvia and Russia, while the prevalence of the inhA mutation varies geographically from 3.3% to over 32%.

Specific single mutations associated with INH or RIF resistance may be detected in less than 1 day using PCR amplification of Mycobacterium sp. nucleic acid, such as M. tuberculosis nucleic acid, followed by DNA sequence analysis.

Various methods for sequence analysis of specific, single Mycobacterium sp. mutations have been employed in the art, such as agarose gel electrophoresis. This method requires mutation specific amplification—the size of the resultant PCR product in a gel indicating the presence of any given mutation. Another method for sequence analysis is SSCP (single strand conformation polymorphism analysis), in which a region of DNA containing a given mutation is amplified by PCR, then this PCR product is denatured, and the resultant single stranded DNA is passed down an acrylamide gel—a typical migration pattern being seen with each mutation. A further technique for sequence analysis is melting curve analysis. Melt curves can be generated using Real-time PCR equipment such as a LightCycler (Roche), and each mutation will have a typical curve. Mutations in PCR products can also be identified using fluorescent probes. Nucleic acid sequences can also be determined using commercially available sequencing equipment.

A disadvantage of all the above sequencing techniques is that they cannot be multiplexed to a degree that allows all DNA analysis to take place in a single test. Although it may, theoretically, be possible to identify a number of mutant sites using a LightCycler PCR assay, in practice there are problems caused by cross-talk when a number of different dyes are used. Hence these known methods do not enable all of the mutant target sites to be identified simultaneously.

An alternative method for sequence analysis is reverse hybridisation. A labelled PCR product is generated that includes the mutation of interest, and this is used to interrogate a series of probes immobilised on a solid support. A system for detecting mutations in rpoB associated with RIF resistance is commercially available (INNO-LiPA Rif.TB, Innogenetics, Gent, Belgium). The application of this kit for screening purposes is, however, limited in regions with high TB incidence and high rates of MDRTB, due to a relatively high cost and impossibility to analyse INH resistance.

Non-commercial dot-blot strategies, based on amplification of gene fragments known to confer INH resistance or RIF resistance, followed by hybridisation with mutant and wild-type oligonucleotide probes, have been found to be a more cost-effective methodology, predicting RIF resistance in 90% of cases or INH resistance in 75% of cases.

There is, therefore, a need to provide an alternative and/or improved system for detecting multi-drug resistant Mycobacterium sp., in particular those members of the MTC, such as multi-drug resistant M. tuberculosis (MDRTB). This need is fulfilled by the present invention, which solves one or more of the above defined technical problems.

Accordingly, a first aspect of the present invention provides a set of nucleic acid probes for use in an assay for detecting multi-drug resistant Mycobacterium sp. in a sample, which set includes probe 1 comprising a nucleic acid sequence of 10 nucleotides that binds to a first target sequence ACCAGCGGCA [SEQ ID NO:39], or to the complement thereof; probe 2 comprising a nucleic acid sequence of 10 nucleotides that binds to a second target sequence GCCGGTGGTG [SEQ ID NO:40], or to the complement thereof; probe 3 comprising a nucleic acid sequence of 10 nucleotides that binds to a third target sequence TATCGTCTCG [SEQ ID NO:41], or to the complement thereof; probe 4 comprising a nucleic acid sequence of 10 nucleotides that binds to a fourth target sequence TATCATCTCG [SEQ ID NO:42], or to the complement thereof; probe 5 comprising a nucleic acid sequence of 10 nucleotides that binds to a fifth target sequence GAATTGGCTC [SEQ ID NO:43], or to the complement thereof; probe 6 comprising a nucleic acid sequence of 10 nucleotides that binds to a sixth target sequence CTGGTCCATG [SEQ ID NO:44], or to the complement thereof; probe 7 comprising a nucleic acid sequence of 10 nucleotides that binds to a seventh target sequence GGTTGTTCTG [SEQ ID NO:45], or to the complement thereof; probe 8 comprising a nucleic acid sequence of 10 nucleotides that binds to an eighth target sequence CCCGACAGCG [SEQ ID NO:46], or to the complement thereof; probe 9 comprising a nucleic acid sequence of 10 nucleotides that binds to a ninth target sequence GCTTGTGGGT [SEQ ID NO:47], or to the complement thereof; probe 10 comprising a nucleic acid sequence of 10 nucleotides that binds to a tenth target sequence CCAGTGCCGA [SEQ ID NO:48], or to the complement thereof; and wherein, once a probe is bound to a respective target sequence, a detectable signal is provided.

It is preferred that each probe comprises a nucleic acid sequence of 15 nucleotides. Thus, in one embodiment, probe 1 comprises a nucleic acid sequence of 15 nucleotides that binds to a first target sequence ATCACCAGCGGCATC [SEQ ID NO:49], or to the complement thereof; probe 2 comprises a nucleic acid sequence of 15 nucleotides that binds to a second target sequence GATGCCGGTGGTGTA [SEQ ID NO:50], or to the complement thereof; probe 3 comprises a nucleic acid sequence of 15 nucleotides that binds to a third target sequence ACCTATCGTCTCGCC [SEQ ID NO:51], or to the complement thereof; probe 4 comprises a nucleic acid sequence of 15 nucleotides that binds to a fourth target sequence ACCTATCATCTCGCC [SEQ ID NO:52], or to the complement thereof; probe 5 comprises a nucleic acid sequence of 15 nucleotides that binds to a fifth target sequence ATGAATTGGCTCAGC [SEQ ID NO:53], or to the complement thereof; probe 6 comprises a nucleic acid sequence of 15 nucleotides that binds to a sixth target sequence GTTCTGGTCCATGAA [SEQ ID NO:54], or to the complement thereof; probe 7 comprises a nucleic acid sequence of 15 nucleotides that binds to a seventh target sequence GCGGGTTGTTCTGGT [SEQ ID NO:55], or to the complement thereof; probe 8 comprises a nucleic acid sequence of 15 nucleotides that binds to an eighth target sequence AACCCCGACAGCGGG [SEQ ID NO:56], or to the complement thereof; probe 9 comprises a nucleic acid sequence of 15 nucleotides that binds to a ninth target sequence GGCGCTTGTGGGTCA [SEQ ID NO:57], or to the complement thereof; probe 10 comprises a nucleic acid sequence of 15 nucleotides that binds to a tenth target sequence GCCCCAGTGCCGACA [SEQ ID NO:58], or to the complement thereof.

The present invention thus relates to the use of a carefully selected set of probes that enable mutations in three target genes (katG, inhA and rpoB) to be detected simultaneously in a single assay. Partial gene sequences for the wild-type version of each of these genes are provided as Genbank accession NOs. MTU06270, MTU66801 and Z95972, respectively.

Of the set of probes provided by the present invention, probes 1 and 2 target the first gene for INH resistance (katG), probes 3 and 4 target the second gene for isoniazid resistance (inhA), and probes 5-10 form a scanning array for an 81 base pair region within rpoB associated with RIF resistance. The probes of the present invention have been optimised both individually and as a group—each being highly specific, enabling individual base mutations to be detected.

The probes of the present invention have been carefully designed to bind to the target gene sequence based on a selection of desired parameters. It is preferred that the binding conditions are such that a high level of specificity is provided—ie. binding occurs under “stringent conditions”. In general, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence binds to a perfectly matched probe. In this regard, the Tₘ of each probe of the present invention, at a salt concentration of about 0.02M or less at pH 7, is preferably above 60° C., more preferably about 70° C. Premixed binding solutions are available (eg. EXPRESSHYB Hybridisation Solution from CLONTECH Laboratories, Inc.), and binding can be performed according to the manufacturer's instructions. Alternatively, one of a skill in the art can devise variations of these binding conditions.

Following binding, the nucleic acid molecules can be washed to remove unbound nucleic acid molecules, under stringent (preferably highly stringent) conditions. Typical stringent washing conditions include washing in a solution of 0.5-2×SSC with 0.1% SDS at 55-65° C. Typical highly stringent washing conditions include washing in a solution of 0.1-0.2×SSC with 0.1% SDS at 55-65° C. A skilled person can readily devise equivalent conditions for example, by substituting SSPE for the SSC in the wash solution.

It is preferable to screen the probes to minimise self-complementarity and dimer formation (probe-probe binding). Preferred probes of the present invention are selected so as to have minimal homology with human DNA. The selection process may involve comparing a candidate probe sequence with human DNA and rejecting the probe if the homology is greater than 50%. The aim of this selection process is to reduce annealing of probe to contaminating human DNA sequences and hence allow improved specificity of the assay.

In one embodiment, the present invention provides a set of probes, which set includes probe 1 comprising the sequence TGCCGCTGGT [SEQ ID NO:59], or a sequence having at least 90% sequence identity thereto; probe 2 comprising the sequence CACCACCGGC [SEQ ID NO:60], or a sequence having at least 90% sequence identity thereto; probe 3 comprising the sequence CGAGACGATA [SEQ ID NO:61], or a sequence having at least 90% sequence identity thereto; probe 4 comprising the sequence CGAGATGATA [SEQ ID NO:62], or a sequence having at least 90% sequence identity thereto; probe 5 comprising the sequence GAGCCAATTC [SEQ ID NO:63], or a sequence having at least 90% sequence identity thereto; probe 6 comprising the sequence CATGGACCAG [SEQ ID NO:64], or a sequence having at least 90% sequence identity thereto; probe 7 comprising the sequence CAGAACAACC [SEQ ID NO:65], or a sequence having at least 90% sequence identity thereto; probe 8 comprising the sequence CGCTGTCGGG [SEQ ID NO:66], or a sequence having at least 90% sequence identity thereto; probe 9 comprising the sequence ACCCACAAGC [SEQ ID NO:67], or a sequence having at least 90% sequence identity thereto; probe 10 comprising the sequence TCGGCACTGG [SEQ ID NO:68], or a sequence having at least 90% sequence identity thereto; wherein the underlined nucleotides within the sequences of probes 1, 2,3 and 4 are essential, and may not be substituted by any other nucleotide.

In a preferred embodiment, the present invention provides a set of 10 probes, which set includes probe 1 comprising the sequence GATGCCGCTGGTGAT [SEQ ID NO:69], or a sequence having at least 90% sequence identity thereto; probe 2 comprising the sequence ATCACCACCGGCATC [SEQ ID NO:70], or a sequence having at least 90% sequence identity thereto; probe 3 comprising the sequence GGCGAGACGATAGGT [SEQ ID NO:71], or a sequence having at least 90% sequence identity thereto; probe 4 comprising the sequence GGCGAGATGATAGGT [SEQ ID NO:72], or a sequence having at least 90% sequence identity thereto; probe 5 comprising the sequence AGCTGAGCCAATTCATG [SEQ ID NO:73], or a sequence having at least 90% sequence identity thereto; probe 6 comprises the sequence AATTCATGGACCAGAACA [SEQ ID NO:74], or a sequence having at least 90% sequence identity thereto; probe 7 comprising the sequence ACCAGAACAACCCGC [SEQ ID NO:75], or a sequence having at least 90% sequence identity thereto; probe 8 comprising the sequence ACCCGCTGTCGGGGTT [SEQ ID NO:76], or a sequence having at least 90% sequence identity thereto; probe 9 comprising the sequence TGACCCACAAGCGCC [SEQ ID NO:77], or a sequence having at least 90% sequence identity thereto; probe 10 comprising the sequence CTGTCGGCACTGGGGCC [SEQ ID NO:78], or a sequence having at least 90% sequence identity thereto; wherein the underlined nucleotides within the sequences of probes 1, 2, 3 and 4 are essential, and may not be substituted by any other nucleotide.

Each probe is preferably 18 to 25 nucleotides in length. Particularly good results have been obtained using a set of 10 probes comprising one of each of probe SEQ ID NOs 1-10 (see Table 1 below).

The present invention thus provides, in a preferred embodiment, a set of probes, which set includes probe 1 comprising the sequence SEQ ID NO:1, or a sequence having at least 90% sequence identity thereto; probe 2 comprising the sequence SEQ ID NO:2, or a sequence having at least 90% sequence identity thereto; probe 3 comprising the sequence SEQ ID NO:3, or a sequence having at least 90% sequence identity thereto; probe 4 comprising the sequence SEQ ID NO:4, or a sequence having at least 90% sequence identity thereto; probe 5 comprising the sequence SEQ ID:5, or a sequence having at least 90% sequence identity thereto; probe 6 comprising the sequence SEQ ID NO:6, or a sequence having at least 90% sequence identity thereto; probe 7 comprising the sequence SEQ ID NO:7, or a sequence having at least 90% sequence identity thereto; probe 8 comprising the sequence SEQ ID NO:8, or a sequence having at least 90% sequence identity thereto; probe 9 comprising the sequence SEQ ID NO:9, or a sequence having at least 90% sequence identity thereto; probe 10 comprising the sequence SEQ ID NO:10, or a sequence having at least 90% sequence identity thereto; wherein the underlined residues within SEQ ID NOs:1, 2, 3 and 4 are essential and may not be substituted by any other nucleotide.

TABLE 1

| SEQ ID | PROBE | PROBE | SEQUENCE OF |
|---|---|---|---|
| NO: | NUMBER | NAME | PROBE |
| 1 | 1 | K315WTC10T | ctc gat gcc gct ggt |
| | | | gat cgc |
| 2 | 2 | K315GC10T | gcg atc acc acc ggc |
| | | | atc gag |
| 3 | 3 | tomiwt10T | ggc gag acg ata ggt |
| | | | tgt cgg |
| 4 | 4 | tomimut110T | ggc gag atg ata ggt |
| | | | ttg cgg |
| 5 | 5 | MRURP3 | gcc agc tga gcc aat |
| | | | tca tgg ac |
| 6 | 6 | MRURP615T | gcc aat tca tgg acc |
| | | | aga aca acc |
| 7 | 7 | MRURP9 | tgg acc aga aca acc |
| | | | cgc tgt c |
| 8 | 8 | MRURP12 | aca acc cgc tgt cgg |
| | | | ggt tga c |
| 9 | 9 | MRURP17 | ggt tga ccc aca agc |
| | | | gcc gac |
| 10 | 10 | MRU1371A | cga ctg tcg gca ctg |
| | | | ggg ccc gg |

Where sequences having “at least 90% sequence identity” to a sequence of the present invention are referred to in the present description, the present invention also embraces probe sequences that have preferably at least 95% sequence identity, more preferably at least 98% sequence identity, most preferably at least 99% sequence identity to probe sequences of the present invention.

Probe sequences having at least 90% sequence identity, preferably at least 95% sequence identity, more preferably at least 98% sequence identity, most preferably at least 99% sequence identity to probe sequences of the present invention may be identified by sequence alignments using conventional software, for example the Bioedit™ package, available free online, and the Sequencher™ package, provided by Sequencher Gene Codes Corporation, 640 Avis Drive Suite 310, Ann Arbor Mich. 48108.

An alternative means for defining probe sequences that are homologous to probe sequences of the present invention is by defining the number of nucleotides that differ between the homologous sequence and the sequence of the invention. In this regard, the present invention embraces probe sequences that differ from the probe sequences of the invention by no more than 5 nucleotides, preferably by no more than 4 nucleotides, more preferably by no more than 3 nucleotides, yet more preferably by no more than 2 nucleotides, and most preferably by no more than 1 nucleotide. The underlined nucleotides in the sequences of probes 1, 2, 3 and 4 must not, however, be substituted by any other nucleotide.

In the present invention, a “complement” or “complementary strand” means the non-coding (anti-sense) nucleic acid strand, which may bind via complementary base-pairing to a coding strand. Hence, the present invention also embraces use of the complements of the probes described herein. By way of example, the complement of Probe 1, above, has the sequence GAG CTA CGG CGA CCA CTA GCG [SEQ ID NO:79] and the complement of probe 2, above, has the sequence CGC TAG TGG TGG CCG TAG CTC [SEQ ID NO:80]. It is well known in the art to work out the sequence of a complementary strand by using the complementary base-pairing rules, if the sequence of the coding strand is known.

In one embodiment of the present invention, the probes may be immobilised onto a solid support or platform. The support may be a rigid solid support made from, for example, glass or plastic, or else the support may be a nylon or nitrocellulose membrane, or other membrane. 3D matrices are suitable supports for use with the present invention—eg. polyacrylamide or PEG gels. In one embodiment, the solid support may be in the form of beads, which may be sorted by size or fluorophores.

The probes may be immobilised to the solid support by a variety of means. By way of example, probes may be immobilised onto a nylon membrane by UV cross-linking. Biotin-labelled probes may be bound to streptavidin-coated substrates, and probes prepared with amino linkers may be immobilised onto silanised surfaces. Another means of immobilising probe is via a poly-T tail, preferably at the 3¹ 3′ end. The poly-T tail consists of a run of from 1 to 100 thymine residues added to the probe at the 3¹ 3′ end with a terminal transferase. Preferably, from 1 to 20 thymine residues are added. The poly-T tail is then baked or UV cross-linked onto the solid substrate. Addition of a poly-T tail appears to have two functions. First, the poly-T tail increases the amount of probe that is immobilised onto the solid support. Second, the poly-T tail conforms the probe in such a way as to improve the efficiency of hybridisation. Once a probe of the present invention is bound to a target Mycobacterium sp. (eg. M. tuberculosis) nucleic acid, a detectable signal is provided that may be detected by known means. A detectable signal may be a radioactive signal but is preferably a fluorescent signal (most preferably a change in fluorescence), or a chromogenic signal employing biotin or digoxygenin.

The present invention also provides a method of detecting multi-drug resistant Mycobacterium sp. In a sample, in particular, members of the MTC such as M. tuberculosis. The method comprises contacting a set of probes according to the present invention with a nucleic acid-containing sample, wherein, once a probe is bound to a target Mycobacterium sp. Nucleic acid in the sample, a detectable signal is provided; and detecting said detectable signal.

A sample may be for instance, a food, sewerage or clinical sample. A particular application of the method is for detection of Mycobacterium sp. in a clinical sample. Clinical samples may include broncho-alveolar lavage specimens (BALS), induced sputa, oropharyngeal washes, blood or other body fluid samples.

In the present method, it is preferred that the presence of multi-drug resistant Mycobacterium sp. in said sample is confirmed by detecting a detectable signal provided by probes 2 and 4 and their respective bound target Mycobacterium sp. nucleic acid sequences in the sample; and detecting the absence of a detectable signal provided by probes 1, 3, 5, 6, 7, 8, 9 and 10 and their respective target Mycobacterium sp. nucleic acid sequences.

It is preferred that the present method allows confirmation of the absence of multi-drug resistant Mycobacterium sp. from said sample by detecting a detectable signal provided by probes 1, 3, 5, 6, 7, 8, 9 and 10 and their respective bound target Mycobacterium sp. nucleic acid sequence in the sample; and detecting the absence of a detectable signal provided by probes 2 and 4 and their respective target Mycobacterium sp. nucleic acid sequences.

In this regard, probes 2 and 4 bind to mutant M. tuberculosis nucleic acid. Probe 2 binds to a specific mutated target site within the katG gene, and probe 4 binds to a specific mutated target site within the inhA gene. Probes 2 and 4 only bind to their specific mutated sequence, and do not bind the wild-type target sequence. Binding of probe 2 and/or 4 to M. tuberculosis nucleic acid in the sample therefore indicates that the sample contains nucleic acid having a mutation in katG and/or inhA respectively. The mutations that are detected by probes 2 and 4 are involved in resistance to isoniazid.

Probes 1, 3 and 5-10 bind to wild type M. tuberculosis nucleic acid. Probe 1 binds to a target site within the katG gene—the wild-type version of the sequence bound by probe 2. Probe 3 binds to a target site within the inhA gene—the wild-type version of the sequence bound by probe 4. Hence, binding of probe 1 and/or 3 to nucleic acid in the sample indicates that the sample contains nucleic acid that is wild-type for katG and/or inhA respectively. Probes 1 and 3 only bind the wild-type sequence, and do not bind when their target sequence is mutated.

Probes 5-10 bind only to wild-type M. tuberculosis nucleic acid, within an 81 base pair target region of the wild-type rpoB gene. If the sample contains wild-type rpoB nucleic acid, then all of probes 5-10 will bind. On the other hand, if the nucleic acid in the sample has specific rpoB mutations associated with rifampin resistance, then fewer than 6 rpoB probes will bind.

In more detail, the presence of multi-drug resistant Mycobacterium sp. nucleic acid is confirmed in the sample by detecting binding of at least one of probes 2 and 4 to mutant nucleic acid (ie. detecting a detectable signal provided by probe 2 and/or 4 and their respective bound target sequences), and detecting non-binding of at least one of probes 1, 3 and at least one of probes 5-10 to wild-type nucleic acid (ie. detecting absence of a detectable signal provided by probes 1, 3 and 5-10 and their respective target sequences).

On the other hand, if probes 2 and 4 do not bind to nucleic acid in the sample (as evidenced by the absence of a detectable signal provided by probes 2 and 4 and their respective target sequences), but both of probes 1, 3 and all of probes 5-10 do bind to nucleic acid in the sample (as evidenced by detection of a detectable signal provided by probes 1, 3 and 5-10 and their respective bound target sequences), this indicates that the sample does not contain multi-drug resistant Mycobacterium tuberculosis nucleic acid.

If a detectable signal is provided by all of the probes and their respective target nucleic acid sequences, this indicates the presence in the sample of Mycobacterium tuberculosis nucleic acid from bacteria that have a wild-type rpoB gene—ie. sensitive to rifampin. This sample also contains nucleic acid from Mycobacterium tuberculosis that have mutant katG and inhA genes—ie. resistant to isoniazid, as well as nucleic acid from Mycobacterium tuberculosis that have wild-type katG and inhA genes—ie. sensitive to isoniazid. Hence, a mixture of INH mono-resistant Mycobacterium tuberculosis and wild-type (RIF/INH sensitive) Mycobacterium tuberculosis are detected.

It is an option to use more than 10 probes—ie. to include further probes in addition to the 10 probes described in detail herein. The further probes may be useful for detection of mutations in other Mycobacterium sp. genes, or for detection of nucleic acids other than Mycobacterium sp. nucleic acid, for example, as part of a wider diagnostic array for analysis of other bacterial species.

In one embodiment, one or more of probes 5-10 described in detail above (SEQ ID NOs: 5-10) may be substituted or used in combination with one or more, preferably 2, 3, 4, 5, 6, 7, 8, 9, 10, 11, 12, 13, 14, 15, 16, 17, 18, 19, 20, 21 or 22 of the probes provided in Table 1a below (probes 11-32, SEQ ID NOs: 11-32). Each of probes 11-32 binds to wild-type M. tuberculosis nucleic acid, within the wild-type rpoB gene. Hence, if the sample contains wild-type rpoB nucleic acid, then probes 11-32 will bind to nucleic acid in the sample, and if the sample contains mutant rpoB nucleic acid, then at least one of probes 11-32 will not bind to nucleic acid in the sample. Thus, probes 11-32 are useful for detecting M. tuberculosis that are resistant to rifampin.

TABLE 1a

| SEQ ID | PROBE | PROBE | SEQUENCE OF |
|---|---|---|---|
| NO: | NUMBER | NAME | PROBE |
| 11 | 11 | MRURP1 | cag cca gcc agc tga gcc aat tc |
| 12 | 12 | MRURP2 | cca gcc agc tga gcc aat tca tg |
| 13 | 13 | MRURP4 | agc tga gcc aat tca tgg acc ag |
| 14 | 14 | MRURP5 | tga gcc aat tca tgg acc aga |
| | | | aca |
| 15 | 15 | MRURP7 | aat tca tgg acc aga aca acc |
| | | | cgc |
| 16 | 16 | MRURP8 | tca tgg acc aga aca acc cgc tg |
| 17 | 17 | MRURP10 | acc aga aca acc cgc tgt cgg g |
| 18 | 18 | MRURP11 | aga aca acc cgc tgt cgg ggt t |
| 19 | 19 | MRURP13 | acc cgc tgt cgg ggt tga ccc |
| 20 | 20 | MRURP14 | cgc tgt cgg ggt tga ccc aca a |
| 21 | 21 | MRURP15 | tgt cgg ggt tga ccc aca agc g |
| 22 | 22 | MRURP16 | cgg ggt tga ccc aca agc gcc |
| 23 | 23 | MRURP18 | tga ccc aca agc gcc gac tgt c |
| 24 | 24 | MRURP19 | ccc aca agc gcc gac tgt cgg |
| 25 | 25 | MRURP20 | aca agc gcc gac tgt cgg cgc |
| 26 | 26 | MRURP21 | agc gcc gac tgt cgg cgc tgg |
| 27 | 27 | MRURP22 | gcc gac tgt cgg cgc tgg ggc |
| 28 | 28 | MRURP23 | gac tgt cgg cgc tgg ggc ccg |

| | | | |
|---|---|---|---|
| 29 | 29 | MRUR24 | tgt cgg cgc tgg ggc ccg gc |
| 30 | 30 | MRURP25 | cgg cgc tgg ggc ccg gcg gt |
| 31 | 31 | MRURP26 | cgc tgg ggc ccg gcg gtc tg |
| 32 | 32 | MRURP27 | tgg ggc ccg gcg gtc tgt cac |

The present invention also embraces probe sequences that have at least 90% sequence identity, preferably at least 95% sequence identity, more preferably at least 98% sequence identity, most preferably at least 99% sequence identity to probe sequences 11-32 of the present invention.

The present assay may be used with or without a prior amplification step, depending on the concentration of M. tuberculosis nucleic acid that is available. Amplification may be carried out by methods known in the art, preferably by PCR.

Preferably, the step of amplifying Mycobacterium sp. nucleic acid in the nucleic acid-containing sample is carried out prior to detection of signal. Most preferably, the step of amplifying Mycobacterium sp. nucleic acid in the sample is carried out prior to contacting the set of probes with the nucleic acid-containing sample.

Amplification of M. tuberculosis nucleic acid is preferably carried out using a pair of sequence specific primers, which bind to a target site within the M. tuberculosis nucleic acid and are extended, resulting in nucleic acid synthesis. Primers of the present invention are designed to bind to the target gene sequence based on the selection of desired parameters, using conventional software, such as Primer Express (Applied Biosystems). In this regard, it is preferred that the binding conditions are such that a high level of specificity is provided. The melting temperature (Tₘ) of the primers is preferably 50° C. or higher, and most preferably about 60° C. The primers of the present invention are preferably screened to minimise self-complementarity and dimer formation (primer-to-primer binding).

The primer pair comprises forward and reverse oligonucleotide primers. A forward primer is one that binds to the complementary, non-coding (anti-sense) strand of the target M. tuberculosis nucleic acid and a reverse primer is one that binds to the coding (sense) strand of the target M. tuberculosis nucleic acid.

The forward and reverse oligonucleotide primers are typically 1 to 50 nucleotides long, preferably 10 to 40 nucleotides long, more preferably 15-25 nucleotides long. It is generally advantageous to use short primers, as this enables faster annealing to target nucleic acid.

Particularly good results have been obtained using the forward (F) and reverse (R) oligonucleotide primers shown in Table 2 below.

TABLE 2

| SEQ ID | | Primer | | F or |
|---|---|---|---|---|
| NO: | Gene | Name | Primer Sequence | R |
| 33 | katG | KatGP5BIO | CGCTGGAGCAGATGGGCTTGG | F |
| 34 | katG | KatGP6BIO | GTCAGCTCCCACTCGTAGCCG | R |
| 35 | inhA | INHAP3BIO | GCAGCCACGTTACGCTCGTGG | F |
| 36 | inhA | TOMIP2BIO | CGATCCCCCGGTTTCCTCCGG | R |
| 37 | rpoB | FTIP1BIO | GGTCGGCATGTCGCGGATGG | F |
| 38 | rpoB | BrpoB1420R | GTAGTGCGACGGGTGCACGTC | R |

It will, however, be appreciated that variants may be employed, which differ from the above-mentioned primer sequences by one or more nucleotides. In this regard, conservative substitutions are preferred. It is also preferred that primers do not differ from the above-mentioned primers at more than 5 nucleotide positions.

It is an option for the probe to be labelled, however, it is preferred that the probe is unlabelled and that, instead, the target Mycobacterium sp. nucleic acid in the sample is labelled. The target nucleic acid may be labelled during PCR amplification, by using labelled primers. Thus, in one embodiment, the target nucleic acid in the sample is labelled and the assay comprises detecting the label and correlating presence of label with presence of Mycobacterium sp. nucleic acid. The label may be a radiolabel but is preferably non-radioactive, such as biotin, digoxygenin or a fluorescence signal such as fluorescein-isothiocyanate (FITC). The label may be detected directly, such as by exposure to photographic or X-ray film, or indirectly, for example, in a two-phase system. An example of indirect label detection is binding of an antibody to the label. In another example, the target nucleic acid is labelled with biotin and is detected using streptavidin bound to a detectable molecule or to an enzyme, which generates a detectable signal. Colorimetric detection systems may also be employed, such as alkaline phosphatase plus NBT/BCIP.

The present invention also provides a single probe selected from the group consisting of: probe 1 comprising the sequence TGCCGCTGGT [SEQ ID NO:59], or the complement thereof; or a sequence having at least 90% sequence identity thereto, or the complement thereof; probe 2 comprising the sequence CACCACCGGC [SEQ ID NO:60], or the complement thereof; or a sequence having at least 90% sequence identity thereto, or the complement thereof; probe 3 comprising the sequence CGAGACGATA [SEQ ID NO:61], or the complement thereof; or a sequence having at least 90% sequence identity thereto, or the complement thereof; probe 4 comprising the sequence CGAGATGATA [SEQ ID NO:62], or the complement thereof; or a sequence having at least 90% sequence identity thereto, or the complement thereof; probe 5 comprising the sequence GAGCCAATTC [SEQ ID NO:63], or the complement thereof; or a sequence having at least 90% sequence identity thereto, or the complement thereof; probe 6 comprising the sequence CATGGACCAG [SEQ ID NO:64], or the complement thereof; or a sequence having at least 90% sequence identity thereto, or the complement thereof; probe 7 comprising the sequence CAGAACAACC [SEQ ID NO:65], or the complement thereof; or a sequence having at least 90% sequence identity thereto, or the complement thereof; probe 8 comprising the sequence CGCTGTCGGG [SEQ ID NO:66], or the complement thereof; or a sequence having at least 90% sequence identity thereto, or the complement thereof; probe 9 comprising the sequence ACCCACAAGC [SEQ ID NO:67], or the complement thereof; or a sequence having at least 90% sequence identity thereto, or the complement thereof; probe 10 comprising the sequence TCGGCACTGG [SEQ ID NO:68], or the complement thereof; or a sequence having at least 90% sequence identity thereto, or the complement thereof; wherein the underlined nucleotides within the sequences of probes 1, 2, 3 and 4 are essential, and may not be substituted by any other nucleotide; for use in an assay for detecting multi-drug resistant Mycobacterium sp. in a sample.

Where sequences having “at least 90% sequence identity” to a sequence of the present invention are referred to in the present description, the present invention also embraces probe sequences that have preferably at least 95% sequence identity, more preferably at least 98% sequence identity, most preferably at least 99% sequence identity to probe sequences of the present invention.

The present invention also provides use of a single probe according to the present invention for the manufacture of a composition for detecting multi-drug resistant Mycobacterium sp. nucleic acid, preferably multi-drug resistant MTC nucleic acid, such as multi-drug resistant M. tuberculosis nucleic acid, in a sample.

Also provided by the present invention is a kit for detection of multi-drug resistant Mycobacterium sp. nucleic acid, preferably multi-drug resistant MTC nucleic acid, such as multi-drug resistant M. tuberculosis nucleic acid, comprising a single probe according to the present invention, or a set of probes according to the present invention.

In accordance with an alternative aspect of the present invention, there is provided an alternative set of nucleic acid probes for use in an assay for detecting multi-drug resistant Mycobacterium sp. in a sample, which set includes probe 1 comprising a nucleic acid sequence of 10 nucleotides that binds to a first target sequence ACCAGCGGCA [SEQ ID NO:39], or to the complement thereof; probe 2 comprising a nucleic acid sequence of 10 nucleotides that binds to a second target sequence GCCGGTGGTG [SEQ ID NO:40], or to the complement thereof; probe 5 comprising a nucleic acid sequence of 10 nucleotides that binds to a fifth target sequence GAATTGGCTC [SEQ ID NO:43], or to the complement thereof; probe 6 comprising a nucleic acid sequence of 10 nucleotides that binds to a sixth target sequence CTGGTCCATG [SEQ ID NO:44], or to the complement thereof; probe 7 comprising a nucleic acid sequence of 10 nucleotides that binds to a seventh target sequence GGTTGTTCTG [SEQ ID NO:45], or to the complement thereof; probe 8 comprising a nucleic acid sequence of 10 nucleotides that binds to an eighth target sequence CCCGACAGCG [SEQ ID NO:46], or to the complement thereof; probe 9 comprising a nucleic acid sequence of 10 nucleotides that binds to a ninth target sequence GCTTGTGGGT [SEQ ID NO:47], or to the complement thereof; probe 10 comprising a nucleic acid sequence of 10 nucleotides that binds to a tenth target sequence CCAGTGCCGA [SEQ ID NO:48], or to the complement thereof; and wherein, once a probe is bound to a respective target sequence, a detectable signal is provided.

This alternative set of probes differs from the set of probes described earlier in that probes 3 and 4 (which target the inhA gene) are not essential. However, this set of probes includes probes 1 and 2, which target the katG gene, and probes 5-10, which target the rpoB gene. Hence, this set of probes is useful for detecting mutations in the rpoB gene (conferring RIF resistance) and the katG gene (conferring INH resistance).

Preferably, this alternative set of probes includes probe 1 comprising the sequence TGCCGCTGGT [SEQ ID NO:59], or a sequence having at least 90% sequence identity thereto; probe 2 comprising the sequence CACCACCGGC [SEQ ID NO:60], or a sequence having at least 90% sequence identity thereto; probe 5 comprising the sequence GAGCCAATTC [SEQ ID NO:63], or a sequence having at least 90% sequence identity thereto; probe 6 comprising the sequence CATGGACCAG [SEQ ID NO:64], or a sequence having at least 90% sequence identity thereto; probe 7 comprising the sequence CAGAACAACC [SEQ ID NO:65], or a sequence having at least 90% sequence identity thereto; probe 8 comprising the sequence CGCTGTCGGG [SEQ ID NO:66], or a sequence having at least 90% sequence identity thereto; probe 9 comprising the sequence ACCCACAAGC [SEQ ID NO:67], or a sequence having at least 90% sequence identity thereto; probe 10 comprising the sequence TCGGCACTGG [SEQ ID NO:68], or a sequence having at least 90% sequence identity thereto; wherein the underlined nucleotides within the sequences of probes 1, 2, 3 and 4 are essential, and may not be substituted by any other nucleotide.

In accordance with this alternative aspect of the invention, there is also provided a method of detecting the presence or absence of multi-drug resistant Mycobacterium sp. in a sample, comprising: (a) contacting the alternative set of probes as described above with a nucleic acid-containing sample wherein, once a probe is bound to Mycobacterium sp. nucleic acid in the sample, a detectable signal is provided; and (b) detecting said detectable signal. Thus, this method allows mutations in the katG and rpoB genes to be detected simultaneously in a single assay.

The present invention also provides a kit for detection of multi-drug resistant Mycobacterium sp. nucleic acid comprising the alternative set of probes as described above. Using this kit, mutations in the rpoB and katG genes may be detected simultaneously in a single assay.

The embodiments of the invention are discussed in more detail by means of the Examples described below. The results referred to in the Examples are illustrated by the accompanying drawings, in which:

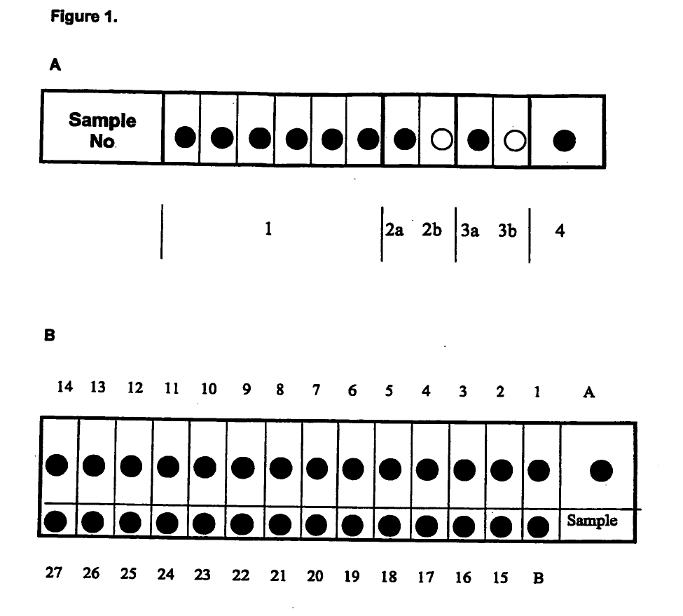

FIG. 1 shows the design of individual macroarrays according to the present invention.

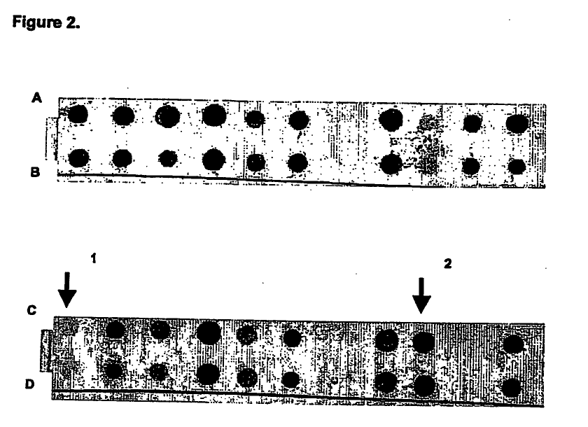

FIG. 2 shows the appearance of developed macroarray membranes.

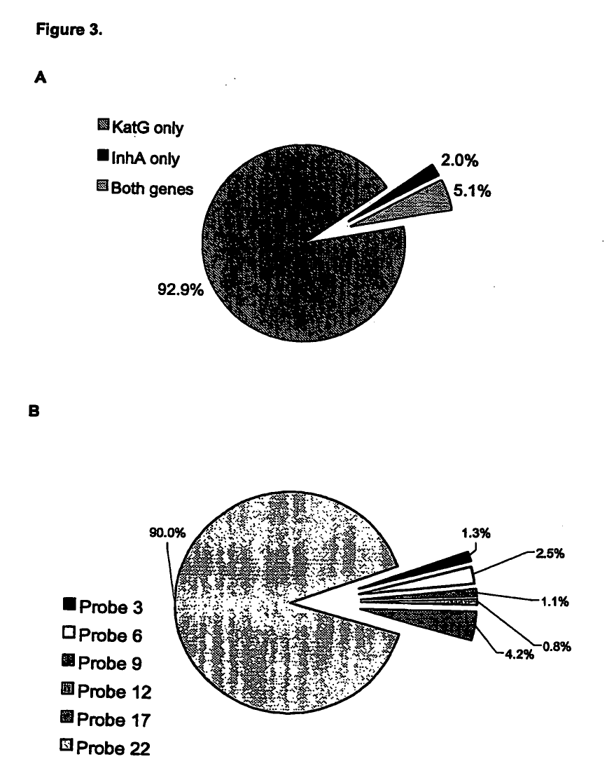

FIG. 3 shows the spectrum of mutations involved in RIF and INH resistance identified in the Samara isolates.

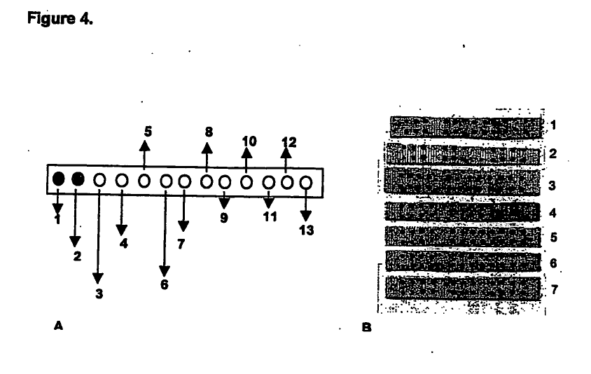

FIG. 4A shows a schematic of the MDR-screen macroarray;

FIG. 4B shows patterns generated by the M. tuberculosis strains.

In more detail, FIG. 1A illustrates a screening macroarray comprising 6 different non-mutant (wild-type) rpoB gene probes (1), non-mutant katG probe (2a), mutant (codon 315) katG gene probe (2b), non-mutant inhA gene probe (3a), mutant (codon 280) inhA gene probe (3b) and a colour development control probe (4).

FIG. 1B illustrates a scanning macroarray, comprising non-mutant (wild-type) rpoB probes (1-27), a colour development control probe (B) and an ink spot (A) for orientation.

FIG. 2A,B illustrates the appearance of a developed macroarray membrane that has been contacted with Mycobacterium tuberculosis wild-type isolates having no mutations in the rpoB, katG or inhA genes (ie. RIF/INH sensitive isolates). Probes 1, 3, and 5-10 have all bound nucleic acid in the sample, indicating the presence of a RIF/INH sensitive genotype.

FIG. 2C, D illustrates the appearance of a developed macroarray membrane that has been contacted with Mycobacterium tuberculosis isolates having mutations in codon 531 of rpoB (1) and codon 315 of katG (2). Fewer than 6 of the rpoB probes are bound, indicating detection of the rpoB mutation, and the mutant katG probe is bound, indicating detection of the katG mutation—thus indicating the presence of a RIF/INH resistant (ie. multi-drug resistant) genotype.

FIG. 3A illustrates the mutations associated with INH resistance in the Samara isolates. 92.9% of mutations are in katG only, with 2.0% of mutations in inhA only, and 5.1% of mutations in both genes.

FIG. 3B illustrates the mutations associated with RIF resistance in the Samara isolates. Probe 3 of the scanning array detected 1.3% of mutations, probe 6 detected 2.5% of mutations, probe 9 detected 1.1% of mutations, probe 12 detected 0.8% of mutations, probe 17 detected 4.2% of mutations and probe 22 detected 90.0% of mutations.

FIG. 4A illustrates a macroarray according to the present invention, as used in Example 4 (below). The key to the spots is as follows:
1ink spot
2 control
3 probe MtbC for detecting probe an M. tuberculosis complex specific locus of M. tuberculosis rpoB
4 probe 1 (of Table 1 above) [SEQ ID NO:1]
5 probe 2 (of Table 1 above) [SEQ ID NO:2]
6 probe 3 (of Table 1 above) [SEQ ID NO:3]
7 probe 4 (of Table 1 above) [SEQ ID NO:4]
8 probe 5 (of Table 1 above) [SEQ ID NO:5]
9 probe 6 (of Table 1 above) [SEQ ID NO:6]
10 probe 7 (of Table 1 above) [SEQ ID NO:7]
11 probe 8 (of Table 1 above) [SEQ ID NO:8]
12 probe 9 (of Table 1 above) [SEQ ID NO:9]
13 probe 10 (of Table 1 above) [SEQ ID NO:10]

FIG. 4B illustrates patterns obtained for the different M. tuberculosis strains:
pattern 1 strain with the katG315 AGC-ACC mutation+rpoB526 mutant allele;
pattern 2 strain with the katG315 AGC-ACC mutation+insertion in the rpoB gene (ins TTC at the 514 codon);
pattern 3 wild type strain;
pattern 4 strain with the rpoB531 mutant allele;
pattern 5 strain with the katG315 AGC-ACA mutation;
pattern 6 strain with the inhA^{C-15T} mutation in the regulatory region of the mabA-inhA operon;
pattern 7 strain with the rpoB516 mutant allele.

### EXAMPLES

### Example 1

### Amplification of Mycobacterium tuberculosis Nucleic Acid

A total of 234 clinical isolates of Mycobacterium tuberculosis from Samara (Central Russia) were available for both phenotypic and genotypic testing.

All sputum specimens were cultured on Lowenstein-Jensen media, and their identity was confirmed using a combination of growth; macroscopic and microscopic appearance; and DNA hybridisation tests. Drug susceptibility testing was performed using the known resistance ratio method.

DNA was extracted by heating cell suspensions with an equal volume of chloroform at +80° C. for 30 minutes, followed by cooling on ice and centrifugation. The upper phase (crude cell lysate) was used for PCR amplification.

Multiplex PCR amplification of regions of the rpoB, katG and inhA genes was carried out using three pairs of primers, followed by dot-hybridization of the amplification products with normal and mutant oligonucleotide probes (single-stranded fragments of rpoB, katG and inhA genes in which mutations occur) immobilized on nylon membrane strips (Osmonics Inc., USA).

In more detail, biotin-labelled PCR products were generated in a multiplex PCR using three pairs of primers. The first pair was for amplification of a fragment including the 81 bp “core” region of the rpoB gene, for detection of mutations consistent with RIF resistance. The second pair was for amplification of a katG gene fragment, including codon 315. The third pair was for the amplification of a inhA gene fragment, including the regulatory region.

PCR was conducted in a 20 μl volume, containing 2 μl 10× PCR buffer (Bioline Ltd., London UK); 0.5 unit Taq-polymerase (Bioline); 0.5 μl 2 mM dNTP mixture (Bioline); 20 μM each of six primers and 1 μl of a DNA extract prepared as described above. Thermal cycling was performed on a Perkin Elmer 9700 Thermocycler using the following amplification programme parameters:

Hold 5 min 95° C.

15 sec 95° C.

30 cycles 30 sec 65° C.

60 sec 72° C.

Hold 5 min 72° C.

The presence of PCR products (3 fragments of 260 bp; 150 bp; 140 bp in length) was detected by electrophoresis in 2.0% agarose gels stained with ethidium bromide. The PCR products were then available for hybridization, with a streptavidin-alkaline phosphatase colour development system being used to visualise the results.

### Example 2

### Hybridisation

The membranes were spotted with oligonucleotide probes using a spotting device (BioGene, UK) in a specific order to produce the arrays (see FIG. 1). After UV cross-linking and washing twice in 0.5×SSC, the membranes were air-dried and cut into separate strips, and placed into 2 ml plastic tubes.

The first array was used to screen the isolates and comprised probes to detect the most frequent mutations in rpoB, katG and inhA genes (see FIG. 1A). For the rpoB gene, the array included 6 non-mutant probes (probe 3 to detect mutations in codons 511, 513 and 514; probes 6 and 9 for codons 513, 514 and 516; probe 12 for codon 516; probe 17 for codon 526 and probe 22 for codon 531). Probes for wild type and the most frequent mutations in the katG gene (AGC→ACC in codon 315) and the regulatory region of the inhA gene (G→T in codon 280) were also included.

A second scanning array was developed to detect other less common mutations in the rpoB gene. A total of 27 non-mutant oligonucleotide probes for the rpoB gene were designed to cover the whole of the 81 bp core region—ie. the sequence in which mutations responsible for rifampin resistance would be found (See FIG. 1B).

Hybridization was performed as follows. Briefly, amplification products were denatured by adding an equal volume of the denaturation solution (0.4M NaOH, 0.02M EDTA) for 15 min at room temperature. In each tube containing an individual array, 500 μl of hybridization solution (5×SSPE; 0.5% SDS) and 20 μl of denatured PCR products were added. Hybridization was performed in rotating tubes in a hybridization oven at 72° C. for 30 min. Membranes were then washed twice in 0.1M Tris-0.1M NaCl solution (pH7.5) and incubated for 1 min in 0.1% Blocking reagent solution (Roche, Mannheim, Germany). After incubation in streptavidin-alkaline phosphatase conjugate solution (1:100) (BioGenex, San Ramon, USA) for 30 min at room temperature and washing, membranes were incubated in 1:250 NBT-BCIP solution (Nitro Blue Tetrasolium, USB, Cleveland, USA, 75 mg/ml in DMF, Bromo-Chloro-Indolil Phosphate, USB, Cleveland, USA, 50 mg/ml in DMF) in a light-proof container for colour development, washed and air-dried.

Sequencing

Sequencing of rpoB and katG gene fragments of selected isolates was performed to verify the results of the macroarray drug susceptibility tests. Template DNA was prepared as described above by chloroform extraction. Amplification products were diluted 1:25 in water and sequenced using a Beckman Coulter SEQ8000 Genetic Analysis System. Sequence data was analysed using SEQ8000 software.

Results

All Samara cultures (except for one, identified as M. fortuitum) were correctly identified as M. tuberculosis, using routine phenotypic tests in the reference laboratory.

Multiplex amplification of 233/234 specimens from the Russian mycobacterial cultures was successful (except for the M. fortuitum). The appearance of the developed membranes is shown in FIG. 2. The results of the macroarray drug susceptibility tests are shown in Table 3 below.

TABLE 3
Results of rifampin and isoniazid resistance detection using a
screening macroarray technique in new cases and chronic cases.

| | New cases | Chronic cases |
|---|---|---|
| Presence of mutations | (n = 78) | (n = 156) |
| Mutations consistent with RIF | 32 (41.0%) | 8 (56.4%) |
| resistance | | |
| (rpoB gene) | | |
| Mutations consistent with | 45 (57.7%) | 22 (78.2%) |
| INH resistance | | |
| (katG and inhA genes) | | |
| Mutations consistent with | 29 (37.2%) | 87 (55.8%) |
| both RIF and INH resistance | | |

In total, 48.7% of isolates possessed mutations consistent with resistance to rifampin, 67.9% of isolates with resistance to isoniazid, and 46.5% of isolates possessed mutations both in rpoB and katG (or inhA) genes. All except one of the M. tuberculosis strains isolated from patients with chronic tuberculosis with mutations consistent with rifampin resistance also possessed mutations consistent with isoniazid resistance.

Results of genotypical (macroarray) and phenotypical drug susceptibility testing were concordant in 90.4% for isoniazid and 79.3% for rifampin resistance. The differences in most cases (in over 60% of cases) were due to phenotypically defined resistance in the absence of mutations associated with resistance identified by the screening macroarray. This suggests that resistance to either RIF or INH may also be associated with other mutations.

We then analysed the spectrum of mutations consistent with resistance to rifampin and isoniazid (see FIG. 3). In over 90% of cases, resistance to INH was due to mutations in codon 315 of katG gene only. In 2.0% of cases, resistance was due to mutations in the inhA gene only, and in 5.1% of cases, both genes contributed to resistance development (see FIG. 3A). Of all isolates carrying mutations consistent with RIF resistance, 90.0% possessed mutations in codon 531, and 4.2% in codon 526 of the rpoB gene (see FIG. 3B). Other mutations (in codons 511-516) were detected in fewer than 6.0% of resistant strains. The second macroarray was designed to detect additional mutations within the 81 bp core region that might be consistent with rifampin resistance in 27 M. tuberculosis DNA specimens that had been previously determined as >wild type=on the screening array (see Table 4).

TABLE 4
Scanning array results of repeated molecular DST for specimens with
disagreements.

| | No of discordant results | |
|---|---|---|
| | Macroarray “wild-type” | Macroarray “mutant” but |
| Scanning array | but phenotypically RIF | phenotypically RIF |
| results | resistant (n = 27) | sensitive (n = 20) |
| Mutant | 20 | 3 |
| Wild-type | 7 | 17 |

Of the 27 M. tuberculosis isolates that had been previously suggested to be wild type, twenty specimens (74.7%) had further mutations consistent with rifampin resistance; but seven samples (25.9%) that were phenotypically resistant did not have any mutations in the core region.

To verify the macroarray drug resistance tests results, rpoB and katG gene fragments were sequenced in selected isolates. RpoB gene sequencing was performed for the seven phenotypically rifampin resistant isolates mentioned above. Two of these isolates were found to possess point mutations in the rpoB gene: one has a CAC→CTC (H→Y) substitution in codon 526 and another has a CTG→CCG (L→P) substitution in codon 533. In another five isolates (4.2% of the total number of rifampin resistant strains) no mutations were detected, suggesting that in these cases rifampin resistance was due to mutations in other genes.

Fragments of the katG gene were sequenced from 6 resistant and 6 sensitive isolates selected according to the macroarray results. All phenotypically sensitive isolates were found to be wild type and possessed no mutations. In the 6 isolates having mutations in the katG gene according to the macroarray, the most common substitution AGC→ACC (S315T) was detected.

Discussion

Combining results from the two macroarrays enabled detection of mutations consistent with resistance in 95.3% of the cultures that were phenotypically rifampin resistant, and in 90.4% of the cultures that were phenotypically isoniazid resistant. These figures for concordance are higher than previously reported for non-commercial molecular drug susceptibility analysis systems, confirming that the inclusion of additional probes into an array increases system performance. Sequence analysis of the rpoB and katG genes proved the macroarray method to be highly specific.

### Example 3

Cultures Studied:

Panel one. 465 INH-resistant strains of M. tuberculosis, with or without concurrent RIF resistance, were used to evaluate the multi-drug resistance (MDR) screen array in a retrospective study. These strains represented all INH-resistant strains from January 1998 to December 2003 referred to the HPA Mycobacterial Reference Unit (MRU).

Panel two. 605 consecutive mycobacterial cultures referred to the HPA MRU for identification and drug susceptibility testing between September and December 2003 were used in a prospective study of the performance of the macroarray.

Mycobacterial cultures were cultured on to Lowenstein-Jensen media or liquid culture media (either MGIT, Becton Dickinson, UK or MB BacT Alert, Biomerieux, Cambridge, UK). Cultures were identified using a combination of microscopic and macroscopic appearance, growth characteristics, biochemical testing and DNA hybridisation (Accuprobe; Genprobe, San Diego, USA). Resistance to isoniazid and rifampicin were determined using the resistance ratio method on Lowenstein-Jensen media.

DNA Extraction

DNA was extracted from mycobacteria using by chloroform extraction. A loop of bacterial culture or 100 μl of liquid culture media was transferred to a microcentrifuge tube and suspended in 100 μl purified water, and an equal volume of chloroform was added. The tubes were heated at 80° C. for 20 minutes, placed in the freezer for 5 minutes, mixed briefly by vortex and centrifuged for 3 minutes at 12000 g just prior to adding to the PCR.

PCR

Target DNA was amplified by PCR using biotinylated primers at the 5′ end to label the PCR products. The reaction mixture of 20 μl contained 5 μl of purified water, 10 μl of 2× reaction buffer, 5 μl of primer mix, 0.2 μl of Taq DNA polymerase (5 units/μl, Bioline) and 1 μl of DNA template. The 2× buffer reaction contained 2.0 ml of 10× Ammonium reaction buffer (NH₄ Bioline), 600 μl of 50 mM Magnesium Chloride (MgCl₂, Bioline), 40 μl of each 100 mM dNTP (dATP, dCTP, dGTP and dTTP, Bioline) and 7240 μl of purified water. The primer mix contained 2.5 μl of primers katPGBIO and katP6BIO (200 μM each), 10 μl of primers inhAP, TomiP2BIO, IP1 (de Beenhouwer et al., 1995) and BrpoB1420R (200 μM each), and 455 μl of purified water.

The amplification reaction was carried out in a DNA thermocycler (GeneAmp PCR System 9700, Applied Biosystems, UK). The thermocycler reaction conditions were 3 min at 95° C., 15 sec at 95° C., 30 sec at 65° C., 60 sec at 72° C. for 30 cycles and a final extension cycle of 5 min at 72° C.

MDR-Screen Macroarray

The macroarray consisted of 11 probes immobilized as spots on a nylon membrane strip (MagnaGraph Nylon Transfer Membrane 0.22 Micron, OSMONICS, USA). The first probe (MRUMtb) was specific for M. tuberculosis complex. The next four probes were designed to detect resistance to isoniazid:—two probes (katGwt and inhAwt) were homologous with the wild-type regions of each gene and two (katGS315T and inhAmut) were homologous with the most frequently seen mutations (the S315T mutation in the katG gene and the inhA^{C-15T} mutation at the 5′ end of a presumed ribosome binding site in the promoter of inhA). The next six probes (P3, P6, P9, P12, P17, and 1371A) were used to detect mutations associated with resistance to rifampicin and constitute the entire 81 bp hypervariable region (RRDR) of the rpoB gene.

The IP1 primer (de Beenhouwer et al., (1995)) was used as a development colour control and Deskjet 690C ink (Hewlett-Packard, UK) was used for the orientation spot.

Hybridization and Colour Detection:

10 μl of PCR products were denatured in 10 μl of Denaturation solution (400 mM NaOH, 20 mM EDTA). The hybridization of PCR-products to nylon strips, containing the immobilized probes, was carried out by incubation of the membranes in 500 μl of hybridization buffer (5×SSPE, 0.5% SDS) at 60° C. for 15 min. The strips were washed in stringent wash buffer (0.4% SSPE, 0.5% SDS) at 60° C. for 10 min. The buffer was discarded and 25 ml of Rinse buffer (0.1M Tris 0.1M NaCl, pH 7.5) were added and agitated for 1 minute in an orbital shaker. Then the buffer was discarded and this step was repeated once.

The strips were incubated for 15 min at room temperature in 5 ml of SAP buffer (Rinse buffer, 0.5% B-M blocking reagent) with 25 μl of alkaline phosphatase conjugated streptavidin (400 μg/ml) to detect the hybridized biotinylated PCR-products. The strips were twice washed in Rinse Buffer and equilibrated in buffer 0.1M Tris, 0.1M NaCl (pH 9.5). Finally, the buffer was discarded and the membranes were incubated in 5 ml of the same buffer containing 15 μl of BCIP (5-bromo-4-chloro-3-indolyl phosphate) and 10 μl of NBT (nitro blue tetrazolium) for 5 minutes. These chromogens served as a substrate for alkaline phosphatase producing a pattern on the strip, which could be interpreted.

Results

Retrospective Study

A total of 465 INH-resistant isolates were analysed by the MDR screen array. Mutations in the regulatory region of the mabA-inhA were identified in 250 isolates (53.8%). Among these isolates the probe inhmut (specifically designed to detect the inhA^{C-15T} mutation) was positive in 240/250 isolates (96.0%). The katGS315T probe (specifically designed to detect the S315T mutation in the katG gene) showed a positive hybridization signal in 161/465 (34.6%) isolates. Both the katGwt and katS315T probes failed to hybridize in 9 (1.9%) isolates. Four (0.9%) isolates showed a positive katGS315T and a negative inhAmut and inhAwt pattern. Forty-one (8.8%) isolates gave a drug susceptible profile.

Prospective Study

The MDR screen was applied to 609 clinical isolates and the results were compared with routine identification and isoniazid and rifampicin susceptibility testing.

Detection of M. Tuberculosis Complex:

From the 609 cultures received for identification and drug susceptibility analysis during the study period, PCR products were obtained from 497 cultures (81.6%). Of these 497 positive reactions, 356 (71.6%) were identified as M. tuberculosis complex, and 141 (28.4%) were identified as non-tuberculosis mycobacteria (NTM) by hybridisation with the macroarray. Of the 356 cultures identified genotypically as M. tuberculosis complex, 353 (99.2%) were confirmed as M. tuberculosis complex by phenotypic examination. Of the 141 genotypically defined NTM, 137 (97.2%) were confirmed phenotypically.

Detection of Susceptible M. Tuberculosis Isolates

Of the 356 isolates identified as M. tuberculosis complex, 289 (81.2%) were identified as M. tuberculosis isolates susceptible to INH and RIF using the macroarray. Two hundred and seventy-seven (95.8%) were concordant with identification and routine susceptibility testing; 3 (1.1%) were phenotypically classified as resistant to INH, and one (0.3%) was phenotypically classified as resistant to RIF.

Detection of M. Tuberculosis rpoB, katG and mabA-inhA Mutants

Of the 356 MTB complex isolates identified, 38 (10.7%) were identified as mono INH-resistant strains, 16 (4.5%) as multi-drug resistant (MTB) strains and 13 (3.6%) as mono RIF-resistant strains. Of the 38 M. tuberculosis isolates resistant to INH by macroarray analysis, 30 (78.9%) were correctly classified according to routine susceptibility testing, 2 (5.3%) were phenotypically classified as MDR-TB, and 5 (13.2%) were phenotypically classified as susceptible M. tuberculosis isolates. Of these 5 isolates, two gave negative hybridization signals for inhAwt and inhAmut probes, one isolate had a positive hybridization signal for katGwt and katGS315T probe, and 2 isolates had a positive signal for the inhAmut probe. The inhAmut probe and the katGS315T were negative for 18 and 15 isolates respectively. Five isolates gave a negative reaction with both probes.

Among the 13 M. tuberculosis isolates resistant to RIF alone, 8 (61.5%) were phenotypically identified as resistant to RIF, 4 (30.8%) were phenotypically identified as MDR-TB and 1 (7.7%) was phenotypically identified as susceptible (a negative hybridization signal for the P3 and P6 probes).

All 16 MDR-TB were phenotypically identified as MDR-TB. Of these 16 isolates, 12 had a positive signal for katGS315T, 1 for inhAmut and 3 for both probes.

Considering all strains defined as either INH or RIF resistant by macroarray, 48/54 (88.9%) were concordant for INH and 28/29 (96.6%) for RIF resistance.

Discussion

The MDR screen macroarray described herein presents a rapid and sensitive method for detecting M. tuberculosis and to determine INH and RIF susceptibility in clinical isolates. The basic principle of the MDR screen array developed in this study is that each nucleotide change should block the hybridization of the target with the corresponding wild-type probes (P3, P6, P9, P17, 1371A, katGwt and inhAwt probes), or permit the hybridization of the target and the corresponding mutant probe (katGS315T and inhAmut probes).

The PCR reaction was positive for detection of M. tuberculosis complex in 356 out of 363 isolates (sensitivity 98.1%) from patients who were later diagnosed by conventional techniques. Eight isolates, classified as M. tuberculosis by routine identification procedures, were not identified by the macroarray. Only 1 isolate out of 356 positive PCR for the M. tuberculosis complex was phenotypically identified as NTM (specificity 99.7%).

The MDR screen macroarray results were concordant with conventional identification and susceptibility testing results for 331 out of 356 M. tuberculosis cases (93.0%) and 137 out of 141 NTM isolates (97.2%). Some of the discrepant isolates displayed wild type array patterns but were phenotypically classified as resistant (3 resistant to isoniazid and one resistant to rifampicin), or they were phenotypically classified as MDR-TB and had a mono-RIF or mono-INH resistant macroarray pattern. Previous studies have demonstrated that approximately 4% of RIF-resistant and 30-40% of INH-resistant isolates have no mutations within the 81-bp region of the rpoB gene (the region associated with RIF resistance) and within the regulatory region of the mabA-inhA operon/within the katG gene, respectively.

More than 95% of RIF-resistant strains are associated with mutations within an 81-bp region of the rpoB gene. The array used in this study is able to detect known mutations, including point mutations, insertions and deletions, because the probes tiled on the array constitute the entire 81 bp wild type hypervariable region. This macroarray has the potential to be used widely as there are no significant differences in the distribution of rpoB mutations globally.

The array used in this study is simple to perform and interpret, requiring only a basic knowledge of molecular biology to perform it successfully. Although DNA sequencing is simple for laboratories already performing it routinely, the costs of equipment and maintenance do not make it a cost-effective option for many clinical laboratories. The cost of our array is an important factor for its widespread applicability. This array advantageously costs less than $5, whereas the commercial INNOLiPA kit costs $720 and only detects resistance to RIF.

The potential of the MDR macroarray for testing different targets has been demonstrated in this study. The array described here can be expanded to detect other specific mutations in the katG gene. Epidemiological markers could also be added to the array for tracing epidemic or sporadic dissemination of strains.

### Example 4

Bacterial Isolates

A panel of 40 M. tuberculosis isolates was assembled in order to give a range of genotypes genotype at the rpoB RRDR, katG315 and mabA-inh⁻¹⁵ loci. These isolates were cultured on LJ and drug susceptibility testing was performed using the resistance ratio method on Lowenstein-Jensen media.

Preparation of DNA Extracts

Cell paste from LJ medium was suspended in 100 μl purified water and an equal volume of chloroform was added. The tubes were heated at 80° C. for 20 minutes, placed in the freezer for 5 minutes and mixed briefly using a vortex mixer. Immediately before use as PCR template tubes were centrifuged for 3 minutes at 12000×g.

PCR

Biotinylated target PCR products were generated in a 20 μl multiplex PCR. This contained 1× Ammonium reaction buffer (Bioline Ltd., London UK), dNTP at 0.2 mM each (Amersham Biosciences, Chalfont St Giles, UK), MgCl₂ at 1.5 mM (Bioline), primers KatGP5IO and KatGP6BIO at 0.25 mM, primers INHAP3BIO, TOMIP2BIO, FTP1BIO and BrpoB142OR at 1 mM (ThermoHybaid, Ulm, Germany), 1 unit Taq-polymerase (Bioline) and 1 μl of DNA template. Primer sequences are given in table 2. Thermal cycling was performed on a Perkin Elmer 9700 Thermocycler using the following program: 5 mins at 95° C., 30× (30 secs at 65° C., 60 secs at 72° C.), hold 5 mins at 72° C.

Construction of MDR Screen Macroarray

The 10 probes illustrated in Table 1, above, were used to produce a macroarray, together with an additional probe that targeted an M. tuberculosis complex specific locus of M. tuberculosis rpoB. Probes 1-4 of Table 1 are designed to analyse loci associated with INH resistance. Probes 1 and 3 (K315WTC and tomiwt), detect the wild-type (WT) genotypes at katG315 and at mabA-inhA⁻¹⁵, whilst Probes 2 and 4 (K315GC and tomimut1), detect the most frequently seen genotype at each locus, katG315^{AGC→ACC} and mabA-inhA^{-15C→T} respectively. Probes 6-10 (MRURP3, MRURP6, MRURP9, MRURP12, MRURP17 and MRU1371A) formed a scanning array for detection of the WT genotype of the RRDR of M. tuberculosis rpoB. In order to optimise probe performance within the array oligonucleotide probes were synthesised with 3′ poly-T tails. Oligonucleotide probes (Invitrogen, Paisley UK) were diluted to 20 μM in water containing 0.001% bromophenol blue and applied to nylon membrane (Magnagraph 0.22 μM, Osmonics, Minnetonka USA) using a hand-held arraying device (VP Scientific, San Diego USA).

In addition to the probes, a permanent ink spot was applied to the membrane in order to orientate the array and a spot of primer FTIP1BIO at 2 μM as a colour development control. Probes were UV-crosslinked to the nylon membrane. The membranes were washed in 0.5% 20×SSC (Sigma, Poole, UK) then dried, cut and placed in 2 ml polythene hybridization tube (Alpha Labs, Eastleigh, UK).

Hybridisation and Colour Detection

The biotin labelled PCR products were denatured by adding an equal volume of denaturation solution (0.4M NaOH, 0.02M EDTA) and incubating at room temperature for 15 minutes. A 20 μl aliquot of the denatured PCR was added to tube containing an array and 500 μl hybridization solution (5×SSPE; 0.5% SDS), which was agitated in a hybridization oven at 60° C. for 15 minutes. The strips were then washed in wash buffer (0.4% SSPE, 0.5% SDS) at 60° C. for 10 min in the hybridization oven. The arrays were now agitated in rinse buffer (0.1M Tris 0.1M NaCl, pH 7.5) at room temperature (RT) for 1 minute. This rinse step was repeated then once more using the rinse buffer containing 0.1% blocking reagent (Roche, Lewes UK). The arrays were now agitated at RT for 15 minutes in the rinse buffer with 0.1% blocking reagent and 1/25 dilution of streptavidin-alkaline phosphatase conjugate at 400 μg/ml (BioGenex, San Ramon USA). The membranes were then washed twice in wash solution and once in substrate buffer (0.1M Tris, 0.1M NaCl at pH 9.5) before being incubated at RT for 5 minutes in substrate buffer containing 0.34 mg/ml NBT (USB, Cleveland, USA) and 0.17 mg/ml BCIP (USB). The membranes were washed in water before being air-dried and the hybridization patterns noted.

Interpretation of the Macroarray

Hybridization to any of the probes directed towards rpoB is indicative of a WT genotype at that locus, conversely lack of hybridization with a given rpoB probe is indicative of a mutant genotype at that locus. Hybridization with K315WTC is indicative of a katG315 WT genotype whereas absence is indicative of a mutant genotype at this or surrounding this locus. Absence of hybridization with K315WTC and hybridization with K315GC is indicative of the katG315 AGC>ACC genotype. Likewise, hybridization with TOMIWT is indicative of a mabA-inhA⁻¹⁵ WT genotype whereas absence is indicative of a mutant genotype at this or surrounding this locus. Absence of hybridization with TOMIWT and hybridization with TOMIMUT1 is indicative of the mabA-inhA^{-15C→T} genotype.

DNA Sequencing

Single primer pairs (see Table 2, above) were used to generate single rpoB, katG or inhA PCR products using the method given above. These were diluted 1/100 in purified water and sequenced using CEQ Quick Start sequencing kits and a CEQ 8000 instrument (Beckman Coulter, High Wycombe, UK) according to the manufacturers instructions. The PCR products were sequenced in both directions using the amplification primers given in Table 2, above.

Results

The panel of 40 M. tuberculosis isolates contained 30 MDR isolates, 5 RIF-mono-resistant isolates, 1 INH mono-resistant isolate and 4 isolates sensitive to RIF and INH. Sequencing of the RRDR of rpoB of these isolates revealed 36 different genotypes in addition to the WT. Analysis of the codons most commonly associated with RIF resistance showed two different mutations at the codon 531, 6 different mutations at the codon 526, and 4 different mutations at the codon 516. Mutations in codons 509, 511, 513, 515, 522, 528, 529 and 533 were also seen. Seven isolates contained two separate single base substitutions, four isolates contained insertions and three contained deletions. The katG315 and mabA-inhA⁻¹⁵ genotype of 28 of the isolates were determined. Three genotypes in addition to the WT were seen at katG315 and a C to T substitution at mabA-inhA⁻¹⁵ was seen in addition to the WT. The genotypes of individual isolates are shown in Table 5, below.

TABLE 5

| | | | katG315/ | | | | |
|---|---|---|---|---|---|---|---|
| | Susceptibility | | mabA- | Non- | | Susceptibility | |
| | by phenotype | | inha-15 | hybridizing | | by array | |
| Isolate | INH | RIF | genotype | rpoB genotype | probes | INH | RIF |
| 01/07786 | R | R | | 1302C > G/S509R = | P2 P5 P10 | R | R |
| | | | | 1351C > T/H526Y | | | |
| 236-02 | R | R | AGC > ACC/ | 1307T > C/L511P = | P2 P5 P8 P7 | R | R |
| | | | WT | 1322A > G/D516G | | | |
| 98/05219 | S | R | | 1307T > C/L511P = | P3 P5 P6 | S | R |
| | | | | 1351C > G/H526D | P10 | | |
| 2936-99 | R | R | WT/WT | 1312C > A/Q513K | P3 P5 P6 P7 | S | R |
| Is20043 | R | R | | 1313A > C/Q513P | P2 P5 P6 P7 | R | R |
| 98/05844 | R | R | AGC > AAC/ | 1313A >T/Q513L | P2 P3 P5 P6 | R | R |
| | | | WT | | P7 | | |
| 02/07435 | R | R | | 1314 CCAACT ins 513 | P2 P5 P6 P7 | R | R |
| 2651-96 | R | R | AGC >ACC | 1315 TTC ins 514 | P2 P5 P6 P7 | R | R |
| | | | WT | | | | |
| Is14373 | R | R | | 1315-1323 Del | P2 P5 P6 P7 | R | R |
| | | | | TTCATGGAC 514-516 | | | |
| Is11195 | R | R | WT/WT | 1316-1318 Del | P3 P5 P6 P7 | S | R |
| | | | | TCA 514-515 | | | |
| Is14786 | R | R | | 1318A > G/M515V = | P3 P4 P6 P7 | R | R |
| | | | | 1351C > A/H526N | P10 | | |
| 1763-97 | R | R | AGC > AAC/ | 1318Ins ATTCAT 515 | P2 P3 P5 P6 | R | R |
| | | | WT | | P7 | | |
| 98/07530 | R | R | | 1320G > A/M515I | P2 P5 P7 P8 | R | R |
| 2883-97 | R | R | AGC > ACC/ | 1321-26 Del GACCAG | P2 P5 P6 P7 | R | R |
| | | | WT | 516-517 | P8 | | |
| Is11125 | R | R | AGC > ACC/ | 1321-2GA > TT/D516F | P2 P4 P7 | R | R |
| | | | WT | | | | |
| 1579-96 | S | R | WT/WT | 1321G > T/D516Y | P3 P5 P7 | | |
| Is14027 | R | R | | 1322A > G/D516G | P3 P4 P7 | | |
| | | | | | P10 | | |
| 1004-01 | R | R | AGC > ACA/ | 1322A > T/D516V | P5 P7 | | |
| | | | WT | | | | |
| 1071-98 | R | R | AGC > ACC/ | 1322A > T/D516V = | P2 P5 P6 P7 | | |
| | | | WT | 1351C > G/H526D | P8 P10 | | |
| 98/00699 | R | R | | 1334 AGAACAACC ins | P3 P4 | | |
| | | | | 520 | | | |
| 1992-00 | R | R | WT/WT | 1339-40TC > CA/S522Q | P3 P5 P9 | | |
| 1445-01 | R | R | AGC > ACC/ | 1340C > G/S522W | P2 P5 P9 | | |
| | | | WT | | | | |
| 395-98 | R | R | AGC > ACC/ | 1340C > T/S522L | P2 P5 P9 | | |
| | | | WT | | | | |
| 03/02007 | R | R | WT/WT | 1350-1CC > TT/T525 = | P3 P5 P10 | | |
| | | | | 1351C > T/H526Y | | | |
| 2323-02 | R | R | AGC > ACC | 1351-2CA > TG/H526C | P2 P5 P10 | | |
| | | | WT | | | | |
| 1828-00 | R | R | WT/inhA | 1351C > G/H526D | P3 P4 P10 | | |
| | | | C-15T | | | | |
| 2031-02 | S | R | WT/WT | 1351C > T/H526Y | P3 P5 P10 | | |
| 3381-97 | R | R | AGC > ACC/ | 1352A > G/H526R | P2 P5 P10 | | |
| | | | WT | | | | |
| 740-97 | R | R | AGC > ACC/ | 1352A > C/H526P | P2 P5 P10 | | |
| | | | WT | | | | |
| 1810-96 | R | R | AGC > AAC/ | 1352A > T/H526L | P2 P3 P5 | | |
| | | | WT | | P10 | | |
| 01/03682 | S | S | | 1359C > T/R528R | P3 P5 P10 | | |
| 02/06539 | S | R | | 1361G > C/R529P | P3 P5 P10 | | |
| 1255-98 | R | R | WT/inhA | 1363C > A/L530M = | P3 P4 P6 P7 | | |
| | | | C-15T | 1367C > T / S531L | P11 | | |
| 01/11196 | R | R | | 1367C > G/S531W | P2 P5 P11 | | |
| Is5 | R | R | WT/WT | 1367C > T/S531L | P3 P5 P11 | | |
| 02/03056 | S | R | WT/WT | 1373T > C/L533P | P3 P5 P11 | | |
| 03/06044 | S | S | WT/WT | WT | P3 P5 | | |
| 03/04307 | S | S | WT/WT | WT | P3 P5 | | |
| 03/05269 | S | S | WT/WT | WT | P3 P5 | | |
| 1182-01 | R | S | AGC > ACA/ | WT | P5 | | |
| | | | WT | | | | |

The crude DNA extracts from each of the isolates in the panel were analysed using the MDR screen macroarray, the design of which is shown in FIG. 4, as are representative examples of the developed arrays. All isolates produced interpretable hybridization patterns with the array and 39 from the 40 detected mutations when they were present. The one isolate that which failed to give a mutant genotype using the array contained a nine base insertion in the rpoB RRDR. All other isolates were correctly identified as mutant or wild type. A mutation was detected in all 35 of the RIF resistant isolates. A mutation was also detected in a RIF susceptible isolate that did indeed carry a synonymous mutation. The array detected mutations at katG315 or mabA-inhA⁻¹⁵ in twenty-seven out of the 31 INH resistant isolates, the remaining four were wild type at these loci.

Amino acid codons 516, 526 and 531 are the most prevalent codons involved in rifampin resistance. These three codons may be responsible for 80% of RIF-resistant M. tuberculosis cases. All the isolates with mutations in these positions were correctly identified. The rpoB531, rpoB526 and rpo516 mutant alleles showed a negative hybridization signal for their respective probes (see FIG. 4B, patterns 4, 1 and 7).

Others less frequent mutations at the 511, 513, 515, 522, 529 and 533 codons and 6 double single mutations, 3 different deletions and two insertions were correctly identified. Only one strain with an insertion of 9 nucleotides (AGAACAACC) at the codon 520 was incorrectly identified.

Four MDR-resistant isolates showed a wild type pattern for isoniazid resistance with positive hybridization signal for the katGwt and inhA probes: This is possible from the resistance to isoniazid is caused by a variety of mutations at several chromosome loci of M. tuberculosis. Mutations in the katG and the regulatory region of the mabA-inhA operon have not been found in approximately 30-50% of the INH-resistant M. tuberculosis isolates.

All the isolates with known sequences of the part relevant of the katG gene and the regulatory region of the mabA-inhA operon were correctly identified. The INH-resistant M. tuberculosis isolates with different mutations in the 315 amino acid position in the katG gene were correctly identified. The INH-resistant isolates with the S315T mutation had a pattern with a negative hybridization signal for the katGwt and a positive hybridization signal for the katGmut probe (FIG. 1B(2)). Others different mutations (S315 ACA, S315 AAC or S315 AGG) showed a pattern with a negative hybridization signal for both probes (FIG. 1B(5)). The INH-resistant M. tuberculosis isolates with the inhA^{C-15T} mutation showed a negative hybridization signal for the inhAwt probe and a positive hybridization signal for the inhAmut probe (FIG. 1B(6)).

Discussion

Detecting drug resistance in M. tuberculosis isolates by determining genotype is an attractive alternative to conventional phenotypic susceptibility testing because results can be generated within hours with minimal manipulation of live organism. Obviously this approach can only be used where genotypic markers for drug resistance have been identified. This is the case for MDRTB where a range of mutations in the RRDR of rpoB are highly specific to RIF resistant isolates and 2 point mutations, one in katG and one associated with inhA are highly specific to INH resistant isolates. By analysing these 3 different loci MDRTB can be identified. Using macroarray analysis all these loci can be analysed in parallel. We have described such a macroarray-based assay for the detection of MDRTB above. The principle of the MDR screen array assay is that a mutation should impede the hybridization of the target to the relevant WT probe or in the case of the katG315 or inhA loci permit the hybridisation to the corresponding mutant probe. This was capable of detecting 35/36 different mutations in the RRDR of rpoB, 3/3 different mutations at katG315 and 1/1 at inhA⁻¹⁵.

When susceptibility is designated by genotype, there are three sources of discrepancy with the more definitive phenotypic testing. Firstly, a resistant isolate may not contain the marker. According to the literature, this is seen in <5% of RIF resistant isolates and between 10 and 30% of INH resistant isolates using the marker used in this study. This type of discrepancy was seen in 4 INH resistant isolates in the present study. Identifying further markers and including these in the assay would reduce these discrepancies. Secondly, a susceptible isolate may contain synonymous mutation which when detected would lead to the isolate being designated resistant. Any discrepancies caused by synonymous mutations at the loci used in this assay may be reduced by identification of said mutations either by sequencing the mutant loci or by inclusion on the macroarray of probes directed at all possible mutations. One rpoB mutant such as this was seen in the present study. A third source of discrepancy is failure to correctly detect mutations present. This type of discrepancy is minimised in this array by careful selection of the probes used. Because the hybridisation behaviour of a given probe and target combination is difficult to predict, it is essential to validate all probes with potential targets. In the present study only one mutation was not detected. This was a 9 base insertion which had the effect of producing a 3 base mismatch at the 5′ end of the 22 base probe MRURP9 (Probe 7 of Table 1) which presumably did not destabilise the hybridisation duplex sufficiently to prevent the detection of hybridization.

In summary, the above-described MDR-screen macroarray identified M. tuberculosis complex isolates resistant to isoniazid and/or rifampicin, the two most important drugs in the treatment of tuberculosis. The assay is easy to perform and interpret and could be implemented into the routine practices of clinical laboratories although most usefully in areas with a high prevalence of MDR M. tuberculosis.

The above Examples demonstrate the broad applicability of macroarrays comprising probes according to the present invention for the detection of mutations consistent with RIF and INH resistance in Mycobacterium species, in particular, in members of the Mycobacterium tuberculosis complex, such as M. tuberculosis. This system is simple and safe to use, and enables rapid identification of MDRTB. The present assay therefore leads to the earlier institution of appropriate chemotherapy, thereby improving the probability of individual cure and generally improving public health.

### Sequence Listing

A Sequence Listing of the nucleic acid sequences set forth in this document appears on a compact disc filed with the United States Patent and Trademark Office that includes a file named “40181FinalSequenceListing(10-30-06)”, which was created Oct. 30, 2006, and constitutes 83.0 KB; all of which is hereby incorporated by reference.

### REFERENCES

Kruuner A, Hoffner S E, Sillastu H, Danilovits M, Levina K, Svenson S B et. al. Spread of drug-resistant pulmonary tuberculosis in Estonia. J. Clin. Microbiol. 2001; 39:3339-45

World Health Organization, International Union Against Tuberculosis. Anti-tuberculosis drug resistance in the world. Geneva, Switzerland, 2000.

Pablos-Mendez A, Raviglione M C, Laszlo A, et al. Global surveillance for antituberculosis-drug resistance, 1994-1997. N Engl J Med 1998; 338:1641-9

Perelman M I. TB control analysis in Russia in 2001. Problemy tuberculeza. 2003; 2:3-11 (in Russian).

Kruuner A, Sillastu H, Danilovits M, Levina K, Svenson S B Hoffner S E, Kallenius G. et. al. Drug resistant tuberculosis in Estonia. Int. J. Tuberc. Lung. Dis. 1998; 2:130-33

Toungoussova O S, Sandven P, Mariandyshev A O, Nizovtseva N I, Bjune G, Caugant D A. Spread of drug-resistant Mycobacterium tuberculosis strains of the Beijing genotype in the Archangel Oblast, Russia. J Clin Microbiol. 2002; 40:1930-7

Drobnievski F A, Balabanova Y M, Ruddy M, Weldon L, Jeltkova K, Brown T. et al. Rifampin- and Multidrug-resistant tuberculosis in Russian civilians and prison inmates: dominance of the Beijing strain family. Emerg. Infect. Dis. 2002; 8:1320-1326

Mokrousov I, Narvskaya O, Often T, et al. High prevalence of KatG Ser315Thr Substitution among Isoniazid-Resistant Mycobacterium tuberculosis Clinical Isolates from Northwestern Russia, 1996 to 2001. Antimicr Agent Chemother 2002; 46:1417-24

Munsiff S S, Bassoff T, Nivin B, Li J, Sharma A, Bifani P, Mathema B et al. Molecular epidemiology of multidrug-resistant tuberculosis, New York City, 1995-1997. Emerg Infect Dis. 2002; 8:1230-8

Agerton T, Valway S E, Blinkhorn R J, Shilkret K L, Reves R, Schluter W W et al. Spread of strain W, a highly drug-resistant strain of Mycobacterium tuberculosis, across the United States. Clin. Infect. Dis. 1999; 29:85-92

Breathnach A S, De Ruiter A. Holdsworth G M C, Bateman N T, O=Sulliovan D G M Rees P J, et al. An outbreak of multi-drug resistant tuberculosis in a London teaching hospital. J. Hosp. Infect. 1998; 39:111-7

Espinal M A. The global situation of MDR-TB. Tuberculosis 2003; 83:44-51

TB microbiological diagnostics standardization. USSR Ministry of healthcare Order No 558, Aug. 6, 1978. (in Russian).

Rattan A, Kalia A, Ahmad N. Multidrug-resistant Mycobacterium tuberculosis: molecular perspectives. Emerg Infect Dis. 1998; 4:195-209

Bodmer T, Zurcher K, Imboden P, Telenti A. Mutation position and type of substitution in the b-subunit of the RNA polymerase influence in-vitro activity of rifampicins in Rifampicin-resistant Mycobacterium tuberculosis. J. Antimicr. Chemother. 1995; 35:345-48

Fluit A C, Visser M R, Schmitz R J. Molecular Detection of Antimicrobial Resistance. Clin. Microbiol. Rev. 2001; 14:836-71

Musser J M Antimicrobial agent resistance in Mycobacteria: molecular genetic insights. Clin. Micr. Rev. 1995; 8:496-514

Alvarado-Esquivel C, Rossau R, Martinez-Garcia S et al. Characterization of rpoB gene mutations in rifampicin resistant Mycobacterium tuberculosis strains isolated from pulmonary tuberculosis patients at 5 Mexican public hospitals. Rev Invest Clin 2001; 56:526-530

Bártfal Z, Somosk⊕vi A, K⊕dm⊕n C et al. Molecular Characterization of rifampin-resistant isolates of Mycobacterium tuberculosis from Hungary by DNA sequencing and the line probe assay. Clin Microbiol. 2001; 39:3736-39

Zhang Y., Heym B., Allen B. et al. The Catalase-peroxydase Gene and isoniazid Resistance of Mycobacterium tuberculosis. Nature. 1992; 358:591-93

Wilson T M, Collins D M. AhpC, a gene involved in izoniazid resistance of the Mycobacterium tuberculosis complex. Molecular Microbiology. 1996; 19:1025-34

Garcia de Viedma G. Rapid detection of resistance in Mycobacterium tuberculosis: a review discussing molecular approaches. Clin Microbiol Infect. 2002; 9:349-359

van Rie A, Warren R, Mshanga I et al. Analysis for a limited number of gene codons can predict drug resistance of Mycobacterium tuberculosis in a high-incidence community. Clin. Microbiol. 2001; 39:636-641.

Collins C H, Grange J M, Yates M D. Tuberculosis bacteriology. Organization and Practice. 2nd Edition. 1997; Oxford: Butterworth-Heinemann.

Yates M D, Drobniewski F A, Wilson S M Evaluation of a rapid PCR-based epidemiological typing method for routine studies of Mycobacterium tuberculosis J. Clin. Microbiol 2002; 40:712-4.

Ahmad, S., E. Fares, G. F. Araj, T. D. Chugh, and A. S. Mustafa. 2002. Prevalence of S315T mutation within the katG gene in isoniazid-resistant clinical Mycobacterium tuberculosis isolates from Dubai and Beirut. Int. J. Tuberc. Lung Dis. 6: 920-926.

Bakonyte, D., A. Baranauskaite, J. Cicenaite, A. Sosnovskaya, and P. Satakenas. 2003. Molecular characterization of isoniazid-resistant Mycobacterium tuberculosis clinical isolates in Lithuania. Antimicrob. Agents Chemother. 47:2009-20011

Martilla, H., H. Soini, E. Eerola, E. Vyshnevskaya, B. Vyshnevskiy, T. Otten, A. Vasilyef, and M. Viljanen. 1998. A Ser315Thr substitution in KatG is predominant in genetically heterogeneous multidrug-resistant Mycobacterium tuberculosis isolates originating from the St. Petersburg area in Russia. Antimicrob. Agents Chemother. 42:2443-2445.

Tracevska, T., I. Jansone, L. Broka, O. Marga, and V. Baumanis. 2002. Mutations in the rpoB and katG genes leading to drug resistance in Mycobacterium tuberculosis in Latvia. J. Clin. Microbiol. 40:3789-3792.

Narvskaya, O., Otten, T., Limeschenco, E., Sapozhnikova, N., Graschenkova, N., Steklova, L., Nikonova, A., Filipenko, M. L., Mokrousov, I., Vyshnevskiy. B., 2002. Nosocomial outbreak of multidrug-resistant tuberculosis caused by a strain of Mycobacterium tuberculosis W-Beijing family in St. Petersburg Russia. Eur. J. Clin Microbiol. Infect Dis. 21, 596-602.

Davies, P. D., 2003. The worldwide increase in tuberculosis: how demographic changes, HIV, infection and increasing numbers in poverty are increasing tuberculosis. Ann. Med. 35-235-243.

De Beenhouwer, H., Liang, Z., Jannes, G., Mijs, W., Machtelinckx, L., Rossau, R., Traore, H., Portaels, F., 1995. Rapid detection of rifampin resistance in sputum and biopsy specimens from tuberculosis patients by PCR and line probe assay. Tubercle Lung. Dis. 76, 425-430.

Collins C H, Grange J M, Yates M D. Tuberculosis bacteriology. Organization and Practice. 2^{nd} Edition. 1997; Oxford: Butterworth-Heinemann.

Brown, T., Anthony, R., 2000. The addition of low numbers of 3′ thymine bases can be used to improve the hybridization signal of oligonucleotides for use within arrays on nylon supports. J Micro Methods. 42,203-207.

Bifani, P. J., Plikaytis, B., Kapur, V., Stockbauer, K., Pan, X., Lutfey, M. L., Moghazeh, S. L., Eisner, W., Daniel, T. M, Kaplan, M. H., Crawford, J. T., Musser, J. M., Kreiswirth, B. N., 1996. Origin and interstate spread of New York City multidrug-resistant Mycobacterium tuberculosis clone family. JAMA. 275,452-457.

1. A set of nucleic acid probes for use in an assay for detecting multi-drug resistant Mycobacterium sp. in a sample, which set includes subset (c) and one or both of subset (a) and subset (b), wherein:
subset (a) is probe 1 or probe 2;
subset (b) is probe 3 or probe 4; and
subset (c) is one or more of probes 5-32;
wherein further:
probe 1 comprises a nucleic acid sequence of about 10 nucleotides that binds to a first target sequence ACCAGCGGCA [SEQ ID NO:39], or to the complement thereof;
probe 2 comprises a nucleic acid sequence of about 10 nucleotides that binds to a second target sequence GCCGGTGGTG [SEQ ID NO:40], or to the complement thereof;
probe 3 comprises a nucleic acid sequence of about 10 nucleotides that binds to a third target sequence TATCGTCTCG [SEQ ID NO:41], or to the complement thereof;
probe 4 comprises a nucleic acid sequence of about 10 nucleotides that binds to a fourth target sequence TATCATCTCG [SEQ ID NO:42], or to the complement thereof;
probe 5 comprises a nucleic acid sequence of about 10 nucleotides that binds to a fifth target sequence GAATTGGCTC [SEQ ID NO:43], or to the complement thereof;
probe 6 comprises a nucleic acid sequence of about 10 nucleotides that binds to a sixth target sequence CTGGTCCATG [SEQ ID NO:44], or to the complement thereof;
probe 7 comprises a nucleic acid sequence of about 10 nucleotides that binds to a seventh target sequence GGTTGTTCTG [SEQ ID NO:45], or to the complement thereof;
probe 8 comprises a nucleic acid sequence of about 10 nucleotides that binds to an eighth target sequence CCCGACAGCG [SEQ ID NO:46], or to the complement thereof;
probe 9 comprises a nucleic acid sequence of about 10 nucleotides that binds to a ninth target sequence GCTTGTGGGT [SEQ ID NO:47], or to the complement thereof;
probe 10 comprises a nucleic acid sequence of about 10 nucleotides that binds to a tenth target sequence CCAGTGCCGA [SEQ ID NO:48], or to the complement thereof;
probe 11 comprises a nucleic acid sequence of about 23 nucleotides that binds to an eleventh target sequence CAGCCAGCCAGCTGAGCCAATTC [SEQ ID NO:11], or to the complement thereof;
probe 12 comprises a nucleic acid sequence of about 23 nucleotides that binds to a twelfth target sequence CCAGCCAGCTGAGCCAATTCATG [SEQ ID NO:12], or to the complement thereof;
probe 13 comprises a nucleic acid sequence of about 23 nucleotides that binds to a thirteenth target sequence AGCTGAGCCAATTCATGGACCAG [SEQ ID NO:13], or to the complement thereof;
probe 14 comprises a nucleic acid sequence of about 24 nucleotides that binds to a fourteenth target sequence TGAGCCAATTCATGGACCAGAACA [SEQ ID NO:14], or to the complement thereof;
probe 15 comprises a nucleic acid sequence of about 24 nucleotides that binds to a fifteenth target sequence AATTCATGGACCAGAACAACCCGC [SEQ ID NO:15], or to the complement thereof;
probe 16 comprises a nucleic acid sequence of about 23 nucleotides that binds to a sixteenth target sequence TCATGGACCAGAACAACCCGCTG [SEQ ID NO:16], or to the complement thereof;
probe 17 comprises a nucleic acid sequence of about 22 nucleotides that binds to a seventeenth target sequence ACCAGAACAACCCGCTGTCGGG [SEQ ID NO:17], or to the complement thereof;
probe 18 comprises a nucleic acid sequence of about 22 nucleotides that binds to a eighteenth target sequence AGAACAACCCGCTGTCGGGGTT [SEQ ID NO:18], or to the complement thereof;
probe 19 comprises a nucleic acid sequence of about 21 nucleotides that binds to a nineteenth target sequence ACCCGCTGTCGGGGTTGACCC [SEQ ID NO:19], or to the complement thereof;
probe 20 comprises a nucleic acid sequence of about 22 nucleotides that binds to a 20^{th} target sequence CGCTGTCGGGGTTGACCCACAA [SEQ ID NO:20], or to the complement thereof;
probe 21 comprises a nucleic acid sequence of about 22 nucleotides that binds to a 21^{st} target sequence TGTCGGGGTTGACCCACAAGCG [SEQ ID NO:21], or to the complement thereof;
probe 22 comprises a nucleic acid sequence of about 21 nucleotides that binds to a 22^{nd} target sequence CGGGGTTGACCCACAAGCGCC [SEQ ID NO:22], or to the complement thereof;
probe 23 comprises a nucleic acid sequence of about 22 nucleotides that binds to a 23^{rd} target sequence TGACCCACAAGCGCCGACTGTC [SEQ ID NO:23], or to the complement thereof;
probe 24 comprises a nucleic acid sequence of about 21 nucleotides that binds to a 24^{th} target sequence CCCACAAGCGCCGACTGTCGG [SEQ ID NO:24], or to the complement thereof;
probe 25 comprises a nucleic acid sequence of about 21 nucleotides that binds to a 25^{th} target sequence ACAAGCGCCGACTGTCGGCGC [SEQ ID NO:25], or to the complement thereof;
probe 26 comprises a nucleic acid sequence of about 21 nucleotides that binds to a 26^{th} target sequence AGCGCCGACTGTCGGCGCTGG [SEQ ID NO:26], or to the complement thereof;
probe 27 comprises a nucleic acid sequence of about 21 nucleotides that binds to a 27^{th} target sequence GCCGACTGTCGGCGCTGGGGC [SEQ ID NO:27], or to the complement thereof;
probe 28 comprises a nucleic acid sequence of about 21 nucleotides that binds to a 28^{th} target sequence GACTGTCGGCGCTGGGGCCCG [SEQ ID NO:28], or to the complement thereof;
probe 29 comprises a nucleic acid sequence of about 20 nucleotides that binds to a 29^{th} target sequence TGTCGGCGCTGGGGCCCGGC [SEQ ID NO:29], or to the complement thereof;
probe 30 comprises a nucleic acid sequence of about 20 nucleotides that binds to a 30^{th} target sequence CGGCGCTGGGGCCCGGCGGT [SEQ ID NO:30], or to the complement thereof;
probe 31 comprises a nucleic acid sequence of about 20 nucleotides that binds to a 31^{st} target sequence CGCTGGGGCCCGGCGGTCTG [SEQ ID NO:31], or to the complement thereof; and
probe 32 comprises a nucleic acid sequence of about 21 nucleotides that binds to a 32^{nd} target sequence TGGGGCCCGGCGGTCTGTCAC [SEQ ID NO:32], or to the complement thereof.

2. The set of nucleic acid probes according to claim 1, wherein subset (c) includes at least six probes.

3. The set of nucleic acid probes according to claim 2, wherein both subset (a) and subset (b) are included in the set.

4. The set of nucleic acid probes according to claim 3, wherein each of probes 1-32 are between about 10 and about 50 nucleotides long.

5. The set of nucleic acid probes according to claim 4, wherein the set consists of probes 1-10.

6. A set of nucleic acid probes for use in an assay for detecting multi-drug resistant Mycobacterium sp. in a sample, which set includes subset (c) and one or both of subset (a) and subset (b), wherein:
subset (a) is probe 1 or probe 2;
subset (b) is probe 3 or probe 4; and
subset (c) is one or more of probes 5-32;
wherein further:
probe 1 comprises TGCCGCTGGT [SEQ ID NO:59] or GATGCCGCTGGTGAT [SEQ ID NO:69] or a sequence that is at least 80% identical thereto;
probe 2 comprises CACCACCGGC [SEQ ID NO:60] or ATCACCACCGGCATC [SEQ ID NO:70] or a sequence that is at least 80% identical thereto;
probe 3 comprises CGAGACGATA [SEQ ID NO:61] or GGCGAGACGATAGGT [SEQ ID NO:71] or a sequence that is at least 80% identical thereto;
probe 4 comprises CGAGATGATA [SEQ ID NO:62] or GGCGAGATGATAGGT [SEQ ID NO:72] or a sequence that is at least 80% identical thereto;
probe 5 comprises GAGCCAATTC [SEQ ID NO:63] or AGCTGAGCCAATTCATG [SEQ ID NO:73] or a sequence that is at least 80% identical thereto;
probe 6 comprises CATGGACCAG [SEQ ID NO:64] or AATTCATGGACCAGAACA [SEQ ID NO:74] or a sequence that is at least 80% identical thereto;
probe 7 comprises CAGAACAACC [SEQ ID NO:65] or ACCAGAACAACCCGC [SEQ ID NO:75] or a sequence that is at least 80% identical thereto;
probe 8 comprises CGCTGTCGGG [SEQ ID NO:66] or ACCCGCTGTCGGGGTT [SEQ ID NO:76] or a sequence that is at least 80% identical thereto;
probe 9 comprises ACCCACAAGC [SEQ ID NO:67] or TGACCCACAAGCGCC [SEQ ID NO:77] or a sequence that is at least 80% identical thereto;
probe 10 comprises TCGGCACTGG [SEQ ID NO:68] or CTGTCGGCACTGGGGCC [SEQ ID NO:78] or a sequence that is at least 80% identical thereto;
probe 11 comprises CAGCCAGCCAGCTGAGCCAATTC [SEQ ID NO:11] or a sequence that is at least 80% identical thereto;
probe 12 comprises CCAGCCAGCTGAGCCAATTCATG [SEQ ID NO:12] or a sequence that is at least 80% identical thereto;
probe 13 comprises AGCTGAGCCAATTCATGGACCAG [SEQ ID NO:13] or a sequence that is at least 80% identical thereto;
probe 14 comprises TGAGCCAATTCATGGACCAGAACA [SEQ ID NO:14] or a sequence that is at least 80% identical thereto;
probe 15 comprises AATTCATGGACCAGAACAACCCGC [SEQ ID NO:15] or a sequence that is at least 80% identical thereto;
probe 16 comprises TCATGGACCAGAACAACCCGCTG [SEQ ID NO:16] or a sequence that is at least 80% identical thereto;
probe 17 comprises ACCAGAACAACCCGCTGTCGGG [SEQ ID NO:17] or a sequence that is at least 80% identical thereto;
probe 18 comprises AGAACAACCCGCTGTCGGGGTT [SEQ ID NO:18] or a sequence that is at least 80% identical thereto;
probe 19 comprises ACCCGCTGTCGGGGTTGACCC [SEQ ID NO:19] or a sequence that is at least 80% identical thereto;
probe 20 comprises CGCTGTCGGGGTTGACCCACAA [SEQ ID NO:20] or a sequence that is at least 80% identical thereto;
probe 21 comprises TGTCGGGGTTGACCCACAAGCG [SEQ ID NO:21] or a sequence that is at least 80% identical thereto;
probe 22 comprises CGGGGTTGACCCACAAGCGCC [SEQ ID NO:22] or a sequence that is at least 80% identical thereto;
probe 23 comprises TGACCCACAAGCGCCGACTGTC [SEQ ID NO:23] or a sequence that is at least 80% identical thereto;
probe 24 comprises CCCACAAGCGCCGACTGTCGG [SEQ ID NO:24] or a sequence that is at least 80% identical thereto;
probe 25 comprises ACAAGCGCCGACTGTCGGCGC [SEQ ID NO:25] or a sequence that is at least 80% identical thereto;
probe 26 comprises AGCGCCGACTGTCGGCGCTGG [SEQ ID NO:26] or a sequence that is at least 80% identical thereto;
probe 27 comprises GCCGACTGTCGGCGCTGGGGC [SEQ ID NO:27] or a sequence that is at least 80% identical thereto;
probe 28 comprises GACTGTCGGCGCTGGGGCCCG [SEQ ID NO:28] or a sequence that is at least 80% identical thereto;
probe 29 comprises TGTCGGCGCTGGGGCCCGGC [SEQ ID NO:29] or a sequence that is at least 80% identical thereto;
probe 30 comprises CGGCGCTGGGGCCCGGCGGT [SEQ ID NO:30] or a sequence that is at least 80% identical thereto;
probe 31 comprises CGCTGGGGCCCGGCGGTCTG [SEQ ID NO:31] or a sequence that is at least 80% identical thereto; and
probe 32 comprises TGGGGCCCGGCGGTCTGTCAC [SEQ ID NO:32] or a sequence that is at least 80% identical thereto;
wherein the underlined nucleotides within the sequences of probes 1, 2, 3, and 4 may not be substituted by any other nucleotide.

7. The set of nucleic acid probes according to claim 6, wherein subset (c) includes at least six probes.

8. The set of nucleic acid probes according to claim 7, wherein both subset (a) and subset (b) are included in the set.

9. The set of nucleic acid probes according to claim 8, wherein each of probes 1-32 are between about 10 and about 50 nucleotides long.

10. The set of nucleic acid probes according to claim 9, wherein the set consists of probes 1-10.

11. The set of nucleic acid probes according to claim 6, wherein each of probes 1-32 comprises a sequence that is at least 90% identical to the respective sequences.

12. The set of nucleic acid probes according to claim 9, wherein:
probe 1 comprises SEQ ID NO:1, or a sequence having at least 90% sequence identity thereto;
probe 2 comprises SEQ ID NO:2, or a sequence having at least 90% sequence identity thereto;
probe 3 comprises SEQ ID NO:3, or a sequence having at least 90% sequence identity thereto;
probe 4 comprises SEQ ID NO:4, or a sequence having at least 90% sequence identity thereto;
probe 5 comprises SEQ ID NO:5, or a sequence having at least 90% sequence identity thereto;
probe 6 comprises SEQ ID NO:6, or a sequence having at least 90% sequence identity thereto;
probe 7 comprises SEQ ID NO:7, or a sequence having at least 90% sequence identity thereto;
probe 8 comprises SEQ ID NO:8, or a sequence having at least 90% sequence identity thereto;
probe 9 comprises SEQ ID NO:9, or a sequence having at least 90% sequence identity thereto;
probe 10 comprises SEQ ID NO:10, or a sequence having at least 90% sequence identity thereto;
wherein the underlined residues within SEQ ID NOs:1, 2, 3 and 4 may not be substituted by any other nucleotide.

13. The set of nucleic acid probes according to claim 12, wherein the set consists of probes 1-10.

14. The set of nucleic acid probes according to claim 9, wherein said probes are immobilised onto a solid support.

15. The set of nucleic acid probes according to claim 6, wherein said probes each have a 3′ poly-T tail.

16. A method of detecting the presence or absence of multi-drug resistant Mycobacterium sp. in a sample, comprising:
(a) combining a set of nucleic acid probes according to claim 9 and a nucleic acid-containing sample;
(b) incubating the nucleic acid probes and the sample under conditions that (i) promote hybridization of complementary DNA and (ii) generate a detectable signal that is associated with said hybridization between one or more of said nucleic acid probes and its or their respective target Mycobacterium sp. nucleic acid sequence(s); and
(c) detecting said detectable signal;
wherein said probes are immobilised onto a solid support and the detectable signal is associated with the locations of each of the immobilised probes.

17. The method according to claim 16, wherein the presence of multi-drug resistant Mycobacterium sp. in said sample is detected by:
(i) detecting a detectable signal that is associated with hybridization between probe 2 or probe 4 and the respective bound target Mycobacterium sp. nucleic acid sequences; and
(ii) observing the absence of a detectable signal with respect to: probe 1 if probe 2 is employed in the detecting step; probe 3 if probe 4 is employed in the detecting step; and one or more probes of subset (c).

18. The method according to claim 17, wherein the observing step employs at least six probes of subset (c).

19. The method according to claim 17, wherein the detecting step employs probe 2 and probe 4.

20. The method according to claim 16, wherein the absence of multi-drug resistant Mycobacterium sp. from said sample is detected by:
(i) detecting a detectable signal that is associated with hybridization between the respective bound target Mycobacterium sp. nucleic acid sequences in the sample and the following: probe 1 if probe 2 is employed in the observing step; probe 3 if probe 4 is employed in the observing step; and one or more probes of subset (c); and
(ii) observing the absence of a detectable signal with respect to probe 2 or probe 4.

21. The method according to claim 20, wherein the observing step employs probe 2 and probe 4.

22. The method according to claim 16, further comprising the step of amplifying Mycobacterium sp. nucleic acid in the sample.

23. The method according to claim 22, wherein said amplification step comprises the step of labeling the Mycobacterium sp. nucleic acid.

24. The method according to claim 16, wherein said detectable signal is a fluorescent signal.

25. A probe selected from the group consisting of:
probe 1, comprising the sequence TGCCGCTGGT [SEQ ID NO:59] or GATGCCGCTGGTGAT [SEQ ID NO:69] or SEQ ID NO:1, or consisting of the complement thereof; or comprising a sequence having at least 80% sequence identity thereto, or consisting of the complement thereof;
probe 2, comprising the sequence CACCACCGGC [SEQ ID NO:60] or ATCACCACCGGCATC [SEQ ID NO:70] or SEQ ID NO:2, or the complement thereof; or a sequence having at least 80% sequence identity thereto, or the complement thereof;
probe 3, comprising the sequence CGAGACGATA [SEQ ID NO:61] or GGCGAGACGATAGGT [SEQ ID NO:71] or SEQ ID NO:3, or the complement thereof; or a sequence having at least 80% sequence identity thereto, or the complement thereof;
probe 4, comprising the sequence CGAGATGATA [SEQ ID NO:62] or GGCGAGATGATAGGT [SEQ ID NO:72] or SEQ ID NO:4, or the complement thereof; or a sequence having at least 80% sequence identity thereto, or the complement thereof;
probe 5, comprising the sequence GAGCCAATTC [SEQ ID NO:63] or AGCTGAGCCAATTCATG [SEQ ID NO:73] or SEQ ID NO:5, or the complement thereof; or a sequence having at least 80% sequence identity thereto, or the complement thereof;
probe 6, comprising the sequence CATGGACCAG [SEQ ID NO:64] or AATTCATGGACCAGAACA [SEQ ID NO:74] or SEQ ID NO:6, or the complement thereof; or a sequence having at least 80% sequence identity thereto, or the complement thereof;
probe 7, comprising the sequence CAGAACAACC [SEQ ID NO:65] or ACCAGAACAACCCGC [SEQ ID NO:75] or SEQ ID NO:7, or the complement thereof; or a sequence having at least 80% sequence identity thereto, or the complement thereof;
probe 8, comprising the sequence CGCTGTCGGG [SEQ ID NO:66] or ACCCGCTGTCGGGGTT [SEQ ID NO:76] or SEQ ID NO:8, or the complement thereof; or a sequence having at least 80% sequence identity thereto, or the complement thereof;
probe 9, comprising the sequence ACCCACAAGC [SEQ ID NO:67] or TGACCCACAAGCGCC [SEQ ID NO:77] or SEQ ID NO:9, or the complement thereof; or a sequence having at least 80% sequence identity thereto, or the complement thereof;
probe 10, comprising the sequence TCGGCACTGG [SEQ ID NO:68] or CTGTCGGCACTGGGGCC [SEQ ID NO:78] or SEQ ID NO:10, or the complement thereof; or a sequence having at least 80% sequence identity thereto, or the complement thereof;
probe 11, comprising the sequence CAGCCAGCCAGCTGAGCCAATTC [SEQ ID NO:11], or the complement thereof; or a sequence that is at least 80% identical thereto, or the complement thereof;
probe 12, comprising the sequence CCAGCCAGCTGAGCCAATTCATG [SEQ ID NO:12], or the complement thereof; or a sequence that is at least 80% identical thereto, or the complement thereof;
probe 13, comprising the sequence AGCTGAGCCAATTCATGGACCAG [SEQ ID NO:13], or the complement thereof; or a sequence that is at least 80% identical thereto, or the complement thereof;
probe 14, comprising the sequence TGAGCCAATTCATGGACCAGAACA [SEQ ID NO:14], or the complement thereof; or a sequence that is at least 80% identical thereto, or the complement thereof;
probe 15, comprising the sequence AATTCATGGACCAGAACAACCCGC [SEQ ID NO:15], or the complement thereof; or a sequence that is at least 80% identical thereto, or the complement thereof;
probe 16, comprising the sequence TCATGGACCAGAACAACCCGCTG [SEQ ID NO:16], or the complement thereof; or a sequence that is at least 80% identical thereto, or the complement thereof;
probe 17, comprising the sequence ACCAGAACAACCCGCTGTCGGG [SEQ ID NO:17], or the complement thereof; or a sequence that is at least 80% identical thereto, or the complement thereof;
probe 18, comprising the sequence AGAACAACCCGCTGTCGGGGTT [SEQ ID NO:18], or the complement thereof; or a sequence that is at least 80% identical thereto, or the complement thereof;
probe 19, comprising the sequence ACCCGCTGTCGGGGTTGACCC [SEQ ID NO:19], or the complement thereof; or a sequence that is at least 80% identical thereto, or the complement thereof;
probe 20, comprising the sequence CGCTGTCGGGGTTGACCCACAA [SEQ ID NO:20], or the complement thereof; or a sequence that is at least 80% identical thereto, or the complement thereof;
probe 21, comprising the sequence TGTCGGGGTTGACCCACAAGCG [SEQ ID NO:21], or the complement thereof; or a sequence that is at least 80% identical thereto, or the complement thereof;
probe 22, comprising the sequence CGGGGTTGACCCACAAGCGCC [SEQ ID NO:22], or the complement thereof; or a sequence that is at least 80% identical thereto, or the complement thereof;
probe 23, comprising the sequence TGACCCACAAGCGCCGACTGTC [SEQ ID NO:23], or the complement thereof; or a sequence that is at least 80% identical thereto, or the complement thereof;
probe 24, comprising the sequence CCCACAAGCGCCGACTGTCGG [SEQ ID NO:24], or the complement thereof; or a sequence that is at least 80% identical thereto, or the complement thereof;
probe 25, comprising the sequence ACAAGCGCCGACTGTCGGCGC [SEQ ID NO:25], or the complement thereof; or a sequence that is at least 80% identical thereto, or the complement thereof;
probe 26, comprising the sequence AGCGCCGACTGTCGGCGCTGG [SEQ ID NO:26], or the complement thereof; or a sequence that is at least 80% identical thereto, or the complement thereof;
probe 27, comprising the sequence GCCGACTGTCGGCGCTGGGGC [SEQ ID NO:27], or the complement thereof; or a sequence that is at least 80% identical thereto, or the complement thereof;
probe 28, comprising the sequence GACTGTCGGCGCTGGGGCCCG [SEQ ID NO:28], or the complement thereof; or a sequence that is at least 80% identical thereto, or the complement thereof;
probe 29, comprising the sequence TGTCGGCGCTGGGGCCCGGC [SEQ ID NO:29], or the complement thereof; or a sequence that is at least 80% identical thereto, or the complement thereof;
probe 30, comprising the sequence CGGCGCTGGGGCCCGGCGGT [SEQ ID NO:30], or the complement thereof; or a sequence that is at least 80% identical thereto, or the complement thereof;
probe 31, comprising the sequence CGCTGGGGCCCGGCGGTCTG [SEQ ID NO:31], or the complement thereof; or a sequence that is at least 80% identical thereto, or the complement thereof; and
probe 32, comprising the sequence TGGGGCCCGGCGGTCTGTCAC [SEQ ID NO:32], or the complement thereof; or a sequence that is at least 80% identical thereto, or the complement thereof;
wherein the underlined nucleotides within the sequences of probes 1, 2, 3, and 4 may not be substituted by any other nucleotide.

26. The probe of claim 25, wherein the probe is selected from the group consisting of:
probe 1, comprising the sequence TGCCGCTGGT [SEQ ID NO:59], or consisting of the complement thereof; or comprising a sequence having at least 80% sequence identity thereto, or consisting of the complement thereof;
probe 2, comprising the sequence CACCACCGGC [SEQ ID NO:60], or the complement thereof; or a sequence having at least 80% sequence identity thereto, or the complement thereof;
probe 3, comprising the sequence CGAGACGATA [SEQ ID NO:61], or the complement thereof; or a sequence having at least 80% sequence identity thereto, or the complement thereof;
probe 4, comprising the sequence CGAGATGATA [SEQ ID NO:62], or the complement thereof; or a sequence having at least 80% sequence identity thereto, or the complement thereof;
probe 5, comprising the sequence GAGCCAATTC [SEQ ID NO:63], or the complement thereof; or a sequence having at least 80% sequence identity thereto, or the complement thereof;
probe 6, comprising the sequence CATGGACCAG [SEQ ID NO:64], or the complement thereof; or a sequence having at least 80% sequence identity thereto, or the complement thereof;
probe 7, comprising the sequence CAGAACAACC [SEQ ID NO:65], or the complement thereof; or a sequence having at least 80% sequence identity thereto, or the complement thereof;
probe 8, comprising the sequence CGCTGTCGGG [SEQ ID NO:66], or the complement thereof; or a sequence having at least 80% sequence identity thereto, or the complement thereof;
probe 9, comprising the sequence ACCCACAAGC [SEQ ID NO:67], or the complement thereof; or a sequence having at least 80% sequence identity thereto, or the complement thereof; and
probe 10, comprising the sequence TCGGCACTGG [SEQ ID NO:68], or the complement thereof; or a sequence having at least 80% sequence identity thereto, or the complement thereof.

27. The probe according to claim 25, wherein the probe is between about 10 and about 50 nucleotides long.

28. A kit for detection of a nucleic acid associated with multi-drug resistant Mycobacterium sp., comprising a probe according to claim 25.

29. A kit for detection of a nucleic acid associated with multi-drug resistant Mycobacterium sp., comprising a set of probes according to claim 6.

30. A set of nucleic acid probes for use in an assay for detecting multi-drug resistant Mycobacterium sp. in a sample, which set includes:
probe 1 comprising a nucleic acid sequence of 10 nucleotides that binds to a first target sequence ACCAGCGGCA [SEQ ID NO:39], or to the complement thereof;
probe 2 comprising a nucleic acid sequence of 10 nucleotides that binds to a second target sequence GCCGGTGGTG [SEQ ID NO:40], or to the complement thereof;
probe 5 comprising a nucleic acid sequence of 10 nucleotides that binds to a fifth target sequence GAATTGGCTC [SEQ ID NO:43], or to the complement thereof;
probe 6 comprising a nucleic acid sequence of 10 nucleotides that binds to a sixth target sequence CTGGTCCATG [SEQ ID NO:44], or to the complement thereof;
probe 7 comprising a nucleic acid sequence of 10 nucleotides that binds to a seventh target sequence GGTTGTTCTG [SEQ ID NO:45], or to the complement thereof;
probe 8 comprising a nucleic acid sequence of 10 nucleotides that binds to an eighth target sequence CCCGACAGCG [SEQ ID NO:46], or to the complement thereof;
probe 9 comprising a nucleic acid sequence of 10 nucleotides that binds to a ninth target sequence GCTTGTGGGT [SEQ ID NO:47], or to the complement thereof;
probe 10 comprising a nucleic acid sequence of 10 nucleotides that binds to a tenth target sequence CCAGTGCCGA [SEQ ID NO:48], or to the complement thereof.

31. The set of nucleic acid probes according to claim 30, which set includes:
probe 1 comprising the sequence TGCCGCTGGT [SEQ ID NO:59], or a sequence having at least 90% sequence identity thereto;
probe 2 comprising the sequence CACCACCGGC [SEQ ID NO:60], or a sequence having at least 90% sequence identity thereto;
probe 5 comprising the sequence GAGCCAATTC [SEQ ID NO:63], or a sequence having at least 90% sequence identity thereto;
probe 6 comprising the sequence CATGGACCAG [SEQ ID NO:64], or a sequence having at least 90% sequence identity thereto;
probe 7 comprising the sequence CAGAACAACC [SEQ ID NO:65], or a sequence having at least 90% sequence identity thereto;
probe 8 comprising the sequence CGCTGTCGGG [SEQ ID NO:66], or a sequence having at least 90% sequence identity thereto;
probe 9 comprising the sequence ACCCACAAGC [SEQ ID NO:67], or a sequence having at least 90% sequence identity thereto;
probe 10 comprising the sequence TCGGCACTGG [SEQ ID NO:68], or a sequence having at least 90% sequence identity thereto;
wherein the underlined nucleotides within the sequences of probes 1, 2, 3, and 4 may not be substituted by any other nucleotide.

32. A method of detecting the presence or absence of multi-drug resistant Mycobacterium sp. in a sample, comprising:
(a) combining a set of nucleic acid probes according to claim 30 and a nucleic acid-containing sample;
(b) incubating the nucleic acid probes and the nucleic acid-containing sample under conditions that (i) promote hybridization of complementary DNA and (ii) generate a detectable signal that is associated with said hybridization between one or more of said nucleic acid probes and its or their respective target Mycobacterium sp. nucleic acid sequence(s); and
(c) detecting said detectable signal;
wherein said probes are immobilised onto a solid support and the detectable signal is associated with the locations of each of the immobilised probes.

33. A kit for detection of multi-drug resistant Mycobacterium sp. nucleic acid comprising a set of probes according to claim 30.
